SEQUENCE LISTING (17 sequences)

SEQ ID NO: 1 - protein, 19 aa, Homo sapiens
MTTQLLLLLVWVAVVGEAQ

SEQ ID NO: 2 - protein, 15 aa, Homo sapiens
LLVWVAVVGEAQTRI

SEQ ID NO: 3 - protein, 17 aa, Homo sapiens
RTELLNVCMNAKHHKEK

SEQ ID NO: 4 - protein, 15 aa, Homo sapiens
QCRPWRKNACCSTNT

SEQ ID NO: 5 - protein, 18 aa, Homo sapiens
KDVSYLYRFNWNHCGEMA

SEQ ID NO: 6 - protein, 16 aa, Homo sapiens
ACKRHFIQDTCLYECS

SEQ ID NO: 7 - protein, 17 aa, Homo sapiens
LGPWIQQVDQSWRKERV

SEQ ID NO: 8 - protein, 15 aa, Homo sapiens
VDQSWRKERVLNVPL

SEQ ID NO: 9 - protein, 16 aa, Homo sapiens
DCEQWWEDCRTSYTCK

SEQ ID NO: 10 - protein, 17 aa, Homo sapiens
RTSYTCKSNWHKGWNWT

SEQ ID NO: 11 - protein, 16 aa, Homo sapiens
CKSNWHKGWNWTSGFN

SEQ ID NO: 12 - protein, 18 aa, Homo sapiens
WHKGWNWTSGFNKCAVGA

SEQ ID NO: 13 - protein, 16 aa, Homo sapiens
VARFYAAAMSGAGPWA

SEQ ID NO: 14 - protein, 18 aa, Homo sapiens
PWAAWPFLLSLALMLLWL

SEQ ID NO: 15 - DNA, 21 bp, synthetic construct
  source 1..21: mol_type = other DNA
actccctcacatcctcaggtt

SEQ ID NO: 16 - DNA, 22 bp, synthetic construct
  source 1..22: mol_type = other DNA
cttagtgggttagtttcacagc

SEQ ID NO: 17 - DNA, 25 bp, synthetic construct
  source 1..25: mol_type = other DNA
caagtgcacccagcaccagctgatc

(19) United States
(12) Patent Application Publication
(10) Pub. No.: US 2026/0216306 A1
(43) Pub. Date: Jul. 30, 2026

(54) DENDRITIC CELL-BASED VACCINES AND USES THEREOF

(71) Applicants: Mayo Foundation for Medical Education and Research, Rochester, MN (US); BioVentures, LLC, Little Rock, AR (US)

(72) Inventors: Keith L. Knutson, Jacksonville, FL (US); Matthew S. Block, Rochester, MN (US); Allan B. Dietz, Chatfield, MN (US); Michael P. Gustafson, Scottsdale, AZ (US); Martin John Cannon, Little Rock, AR (US)

(21) Appl. No.: 19/148,491

(22) PCT Filed: Feb. 7, 2024
(86) PCT No.: PCT/US2024/014767

§ 371 Date: Jul. 16, 2025

Related U.S. Application Data
(63) Continuation-in-part of application No. PCT/US2024/012603, filed on Jan. 23, 2024.
(60) Provisional application No. 63/443,942, filed on Feb. 7, 2023.
(60) Provisional application No. 63/443,942, filed on Feb. 7, 2023.

(51) Int. Cl. A61K 39/00; A61K 35/15; A61K 38/00; A61P 35/00; C07K 16/00

(52) U.S. Cl. CPC A61K 39/0011; A61K 35/15; A61K 38/00; A61P 35/00; C07K 16/00; A61K 2039/5154; A61K 2039/545; A61K 2039/892; C07K 2317/76

(57) ABSTRACT

Methods and materials for treating cancer (e.g., ovarian cancer) using a combination of a Th17 DC vaccine, optionally in combination with an immune checkpoint inhibitor, are provided herein.

### CROSS-REFERENCE TO RELATED APPLICATIONS

This application claims priority from U.S. Provisional Application Ser. No. 63/443,942, filed Feb. 7, 2023. The disclosure of the prior application is considered part of (and is incorporated by reference in) the disclosure of this application.

### STATEMENT AS TO FEDERALLY SPONSORED RESEARCH

This invention was made with government support under CA136393 awarded by the National Institutes of Health. The government has certain rights in the invention.

### SEQUENCE LISTING

This application contains a Sequence Listing that has been submitted electronically as an XML file named “07039-2177WO2 SL ST26.xlm.” The XML file, created on Feb. 7, 2024, is 15,842 bytes in size. The material in the XML file is hereby incorporated by reference in its entirety.

### TECHNICAL FIELD

This document relates to methods and materials for treating cancer (e.g., ovarian cancer) using one or more vaccine compositions capable of activating T cells within a mammal to form activated IL-17-secreting T cells, optionally in combination with one or more immune checkpoint inhibitors.

### BACKGROUND

Ovarian cancer (OC) causes about 14,000 deaths each year in the United States (Siegel et al., CA Cancer J Clin, 69:7-34, 2019). While there have been significant advances in treatment (e.g., PARP inhibitors, anti-VEGF antibody, neoadjuvant and hyperthermic intraperitoneal chemotherapy), disease progression is common and cure rates remain low (Ledermann, Lancet Oncol 20:470-472, 2019; Mirza et al., Ann Oncol 29:1366-1376, 2018; Killock, Nat Rev Clin Oncol 14:713, 2017; Dizon, Lancet 390:1929-1930, 2017; Colombo et al., Crit Rev Oncol Hematol 97:335-48, 2016; van Driel et al., N Engl J Med 378:1363-1364, 2018; and Wright et al., J Clin Oncol 34:3460-73, 2016). The adaptive and innate immune systems are thought to be important for patient outcome (Li et al., Oncotarget 8:15621-15631, 2017; Santoiemma and Powell, Cancer Biol Ther 16:807-820, 2015; Worzfeld et al., Front Oncol 7:24, 2017; Odunsi, Ann Oncol 28: viii1-viii7, 2017; and Turner et al., Gynecol Oncol 142:349-356, 2016). Despite extensive efforts, however, therapies designed to harness the immune system and prevent ovarian cancer progression have generally been met with limited success. For example, clinical trials have tested immune checkpoint inhibitors (ICIs) either alone or in combination with other agents, largely in the setting of recurrent disease, but the overall response rates have been unimpressive.

### SUMMARY

This document provides methods and materials for treating cancer (e.g., ovarian cancer or breast cancer). For example, this document provides methods and materials for administering, to a mammal having cancer (e.g., ovarian cancer or breast cancer), (a) one or more vaccine compositions capable of activating T cells within a mammal to form activated IL-17-secreting T cells, and optionally (b) one or more agents for immune checkpoint blockade (ICB; such agents are also referred to herein as immune checkpoint inhibitors or ICIs). As described herein, a vaccine composition can be designed to include dendritic cells (DCs) that were exposed to an IL-15 polypeptide and a p38 MAPK inhibitor and that were pulsed with one or more antigens (e.g., one or more antigens expressed by a cancer to be treated). Such a vaccine composition can have the ability to activate T cells within a mammal to form activated IL-17-secreting T cells, and such activated IL-17-secreting T cells can have the ability to recognize one or more antigens expressed by the cancer. For convenience, (a) DCs matured via exposure to at least IL-15 and a p38 MAPK inhibitor can be referred to as Th17 DCs, (b) vaccine compositions that contain Th17 DCs and that are capable of activating T cells within a mammal to form activated IL-17-secreting T cells that can recognize one or more antigens expressed by the cancer to be treated can be referred to as Th17 DC vaccines, and (c) activated IL-17-secreting T cells can be referred to as Th17 T cells.

As demonstrated herein, Th17 DC vaccines can be generated and used to activate T cells to form activated IL-17-secreting T cells in vivo, to restructure the immune microenvironment, and to reduce the likelihood of cancer progression or relapse. Th17 DC vaccines containing antigen-bound murine DCs that can activate Th17 T cells in mice were generated by stimulating the IL-15 pathway while simultaneously blocking the p38 MAPK pathway in otherwise ordinary bone marrow derived DCs, followed by antigen pulsing with tumor cell lysates. Treatment of tumor-bearing mice with the Th17 DC vaccines resulted in increased levels of IL-17 producing T cells in the tumor microenvironment, a restructured myeloid microenvironment, and improved survival of tumor-bearing mice as compared to treatment with DC vaccines containing DCs that were not activated by IL-15 stimulation and p38 MAPK inhibition. Treatment of mice with Th17 DC vaccines resulted in sensitization of ovarian cancer to anti-PD-1 ICB treatment, leading to durable progression free survival by preventing cytokine-mediated resistance. Treatment of mice with Th17 DC vaccines also resulted in sensitization of triple-negative breast cancer to anti-PD-1 ICB treatment, leading to increased survival. Efficacy of the Th17 DC vaccines for treating OC, either alone or in combination with ICB, was CD4 T cell-dependent but did not rely on production of the cytokine IL-17 or induction of CD8 T cell infiltration. These results demonstrate that vaccination with Th17 DC vaccines can overcome resistance to ICB therapy in ovarian tumor-bearing mice by generating new tumor-specific immunity, restructuring the tumor immune microenvironment, and preventing the development of adaptive IL-10-mediated resistance. These results, particularly with regard to the activation and reliance on CD4 T cells, the prevention of adaptive resistance mediated by IL-10, and the synergistic effect of combined treatment with a Th17 DC vaccine and ICB, were surprising.

The results presented herein indicate that biologically relevant immune modifiers, such as Th17 DC vaccines, can be used in cancer treatment (e.g., treatment for OC or breast cancer) to condition the tumor microenvironment for improved clinical responses to ICB therapy. Having the ability to treat a particular cancer (e.g., OC) that typically is refractive to treatment allows clinicians to provide an approach that is more likely to be effective, thereby improving disease-free survival and/or overall survival and/or minimizing subjecting patients to ineffective treatments.

In general, one aspect of this document features methods for treating a mammal having cancer. The method can include, or consist essentially of, administering, to the mammal, an effective amount of a vaccine composition capable of activating T cells within the mammal to form activated IL-17-secreting T cells that can recognize one or more antigens expressed by the cancer. The mammal can be a human. The cancer can be ovarian cancer, melanoma, chronic lymphocytic leukemia, gastric cancer, cervical cancer, colorectal cancer, breast cancer, or lung cancer. The vaccine composition can contain dendritic cells (DCs) presenting one or more folate receptor alpha (FRα) antigens. The one or more FRα antigens can include one or more peptides having the amino acid sequence set forth in SEQ ID NO:3, SEQ ID NO:4, SEQ ID NO:5, SEQ ID NO:7, or SEQ ID NO:14. The method can include administering a dose of about 5×10⁶ to about 50×10⁶ of the DCs composition to the mammal. The method can include administering the vaccine composition to the mammal two or more times over a time period of about 2 weeks to about 16 weeks. The method can further include monitoring the mammal after the administering step to determine an effect of the vaccine composition on the mammal. The monitoring can include using computed tomography, positron emission tomography/computed tomography, bone scan, or magnetic resonance imaging. The monitoring can include measuring the level of one or more biomarkers in a tumor biopsy obtained from the mammal after the administering step, where the one or more biomarkers are selected from the group consisting of CCL6, CX3CL1, CXCL16, GM-CSF, ICAM-1, IL-1a, LIX, osteoprotegerin, and RBP-4. The method can further include administering, to the mammal, an effective amount of an immune checkpoint inhibitor (ICI). The ICI can be selected from the group consisting of pembrolizumab, atezolizumab, nivolumab, durvalumab, avelumab, tremelimumab, ipilimumab, and cemiplimab. The method can include administering a dose of about 50 mg to about 1500 mg of the ICI to the mammal. The method can include administering the ICI to the mammal two or more times over a time period of about 2 weeks to about 16 weeks. The method can further include monitoring the mammal after the administering steps to determine an effect of the vaccine composition and the ICI on the mammal. The monitoring can include using computed tomography, positron emission tomography/computed tomography, bone scan, or magnetic resonance imaging. The monitoring can include measuring the level of one or more biomarkers in a tumor biopsy obtained from the mammal after the administering steps, where the one or more biomarkers are selected from the group consisting of CCL6, CX3CL1, CXCL16, GM-CSF, ICAM-1, IL-1a, LIX, osteoprotegerin, and RBP-4.

In another aspect, this document features methods for treating a mammal having cancer, where the method includes, or consists essentially of: (a) administering, to the mammal, an effective amount of a vaccine composition capable of activating T cells within the mammal to form activated IL-17-secreting T cells that can recognize one or more antigens expressed by the cancer, and (b) administering, to the mammal, an effective amount of an immune checkpoint inhibitor (ICI). The mammal can be a human. The cancer can be ovarian cancer, melanoma, chronic lymphocytic leukemia, gastric cancer, cervical cancer, colorectal cancer, breast cancer, or lung cancer. The vaccine composition can contain dendritic cells (DCs) presenting one or more folate receptor alpha (FRα) antigens. The one or more FRα antigens can include one or more peptides having an amino acid sequence set forth in SEQ ID NO:3, SEQ ID NO:4, SEQ ID NO:5, SEQ ID NO:7, or SEQ ID NO:14. The method can include administering a dose of about 5×10⁶ to about 50×10⁶ of the DCs composition to the mammal. The method can include administering the vaccine composition to the mammal two or more times over a time period of about 2 weeks to about 16 weeks. The ICI can be selected from the group consisting of pembrolizumab, atezolizumab, nivolumab, durvalumab, avelumab, tremelimumab, ipilimumab, and cemiplimab. The method can include administering a dose of about 50 mg to about 1500 mg of the ICI to the mammal. The method can include administering the ICI to the mammal two or more times over a time period of about 2 weeks to about 16 weeks. The method can further include monitoring the mammal after administering steps (a) and (b) to determine an effect of the vaccine composition and the ICI on the mammal. The monitoring can include using computed tomography, positron emission tomography/computed tomography, bone scan, or magnetic resonance imaging. The monitoring can include measuring the level of one or more biomarkers in a tumor biopsy obtained from the mammal after the administering steps (a) and (b), where the one or more biomarkers are selected from the group consisting of CCL6, CX3CL1, CXCL16, GM-CSF, ICAM-1, IL-1a, LIX, osteoprotegerin, and RBP-4.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1M show that IL-15 costimulation and p38 MAPK blockade selectively upregulates expression of MHC class II and IFN-γ while suppressing IL-10 and IL-12 production. FIG. 1A shows a representative western blot of phospho-ATF-2 and β-actin in DCs treated with IL-15 and SB203580 (“SB203580” in FIG. 1A; also referred to herein as Th17 DCs) or without IL-15 and SB203580 (“Control” in FIG. 1A; also referred herein as cDC). FIG. 1B is a graph plotting the mean relative numbers of DCs generated using conventional DC maturation (CDC) or Th17 DC maturation (n=3). FIG. 1C is a graph plotting the mean (+s.e.m., n=3-4) percentage of CD11C⁺ DCs positive for MHC class II, MHC class I, CD80, CD86 and OX40L. DCs were prepared under cDC or Th17 DC conditions and pulsed with or without tumor lysates (Ag). FIG. 1D is a graph plotting the relative mean (+s.e.m., n=3-4) fluorescence intensity indicating surface expression of MHC class II, MHC class I, CD80, CD86 and OX40L on cells derived from FIG. 1C. FIGS. 1E-1J are box and whisker plots showing cell culture supernatant concentrations of IL-10 (FIG. 1E), IL-12 (FIG. 1F), IFN-γ (FIG. 1G), IL-6 (FIG. 1H), IL-1B (FIG. 1I), and TGF-β (FIG. 1J) in DCs stimulated under cDC or Th17 DC conditions in the presence or absence of tumor cell lysates (Ag) (n=4). Each symbol represents a unique replicate. Inset P values above lines and bars were calculated with one-way ANOVAs followed by the Tukey's multiple comparisons test. FIGS. 1K-1M are box and whisker plots showing densitometric units derived from dots blots of IL-13 (FIG. 1K), BAFF (FIG. 1L), and LIX (FIG. 1M) from three independent experiments (n=6). P values were calculated using Mann-Whitney tests. Data shown are representative of three independent experiments.

FIGS. 2A-2B show that Th17 DCs have elevated levels of MHC class II. FIG. 2A includes density plots of MHC class II staining of CD11c⁺ DC matured under cDC or Th17 DC conditions, and with or without tumor antigen (Ag). Inset numbers reflect the percentage of cells that stained for both CD11c and MHC II. FIG. 2B includes cytometry histograms (open histograms) of MHC I, CD80, CD86, and OX-40L. Irrelevant antibody staining is shown in black histograms.

FIG. 3 shows that DC matured under Th17-inducing DC conditions differ in production of selected cytokines. The panel shows a heat map summary of cytokines detected with dot blotting from three independent experiments. P values were calculated using Mann-Whitney tests.

FIG. 4 shows that Th17 DCs phagocytose antigen at a higher rate than cDCs. The bar graph depicts the mean (+s.e.m., n=4) PKH26-labeled cDCs or Th17 DCs following 24-hour exposure to PKH26-labeled tumor cell lysates.

FIGS. 5A-5E show that IL-15 costimulation and p38-MAPK blockade specifically empowers DC vaccines to generate IL-17⁺ T cells and high avidity antibodies in vivo in addition to IFN-γ⁺ and IL-4⁺ T cells. FIGS. 5A-5C include images showing representative ELIspot wells and min/max box and whisker plots depicting the number of antigen-specific IFN-γ⁺ (FIG. 5A), IL-4⁺ (FIG. 5B), and IL-17⁺ (FIG. 5C) T cells per million splenocytes following vaccination with PBS or with cDCs or Th17 DCs that were either pulsed or not pulsed with tumor lysate antigen (Ag) (n=9-18). FIGS. 5D-5E are min/max box and whisker plots showing total tumor (FIG. 5D) and high avidity (FIG. 5E) antigen-specific-IgG antibody levels in the blood (n=12-18). The results shown were derived from three independent experiments. Each symbol represents a unique replicate. P values shown in FIGS. 5A-5C compare Ag+DC and Ag+Th17 DC. P values were calculated with one-way ANOVAs followed by the Tukey's multiple comparisons test (FIGS. 5A-5C) or Fisher's LSD test (FIGS. 5D-5E).

FIGS. 6A-6F show that IL-15 costimulation and p38-MAPK blockade empowers DC vaccines to rapidly generate antigen-specific Th17 T cells in vitro and in vivo. FIGS. 6A and 6B include representative images of ELIspot wells and bar graphs plotting the mean (+s.e.m., n=3) number of antigen-specific IL-17⁺ T cells/well following in vitro DC vaccination with or without SB203580/IL-15 (SB/IL-15) and tumor cell lysate (Ag) at ratios DC/splenocyte ratios of 1:2 (FIG. 6A) and 1:1 (FIG. 6B) for 72 hours. FIG. 6C includes representative images of IL-17 ELIspot analysis wells and bar graphs that depict the mean (+s.e.m., n=3) number of antigen-specific IL-17⁺ T cells per million splenocytes in mice immunized in vivo with or without SB/IL-15 and tumor cell lysate (Ag). FIG. 6D includes representative images showing ELIspot wells and bar graphs plotting the mean (+s.e.m., n=3) number of Th17 T cells per million purified CD4 T cells isolated from splenocytes shown in FIG. 6C. FIG. 6E shows min/max box and whisker plots (n=8) of the levels of IL-17 and IL-10 in the peripheral blood of tumor-bearing mice vaccinated with PBS, non-antigen pulsed cDCs (cDCs), non-antigen Th17 DCs (Th17 DCs), antigen-pulsed cDCs (Ag+cDCs), or antigen-pulsed Th17 DCs (Ag+Th17 DCs) at Day 42 following tumor challenge and vaccination with PBS, cDCs or Th17 DCs pulsed with or without antigen. FIG. 6F is a graph plotting levels of IL-17 in the blood of tumor-bearing mice immunized in vivo with PBS or antigen-pulsed Th17 DC vaccines without or with anti-CD4 or anti-CD8 antibody to deplete CD4 or CD8 T cells, respectively. P values were calculated with one-way ANOVAs followed by the Tukey's multiple comparisons test.

FIG. 7 shows that Th17 DCs induce T cells that generate a unique cytokine profile compared to cDCs following stimulation with antigen presenting cells. The panel shows a heat map summary of cytokines detected with dot blotting following cDC stimulation of purified CD4 T cells derived from splenocytes of mice immunized with PBS, antigen pulsed cDCs, or antigen-pulsed Th17 DCs. Each box represents the median of 6 to 8 replicates. Adjusted P values (far right two columns) were calculated using one-way ANOVA followed by the Tukey's multiple comparisons test. Medians of zero are marked.

FIGS. 8A and 8B show that Th17-inducing vaccines eliminate shedding into the peritoneal cavity. FIGS. 8A-8B show representative Ki67 (FIG. 8A) or SP17 (FIG. 8B) immunohistochemistry (IHC) analysis of peritoneal washings from tumor-bearing mice at Day 42 in mice immunized with PBS, non-antigen-pulsed cDCs (cDCs), non-antigen-pulsed Th17 DCs (Th17 DCs), antigen-pulsed cDCs (Ag+cDCs), and antigen-pulsed Th17 DCs (Ag+Th17 DCs). Brown spots indicate positive staining. The inset graph in each panel plots the mean (+s.e.m., n=3-4) percent of Ki-67⁺ or SP17⁺ cells as a percent of total cells. P values were with calculated using unpaired Student's T tests.

FIGS. 9A-9I show that vaccination induces infiltration of T cells into tumor tissue and extends the lifespan of mice bearing ovarian cancer. FIG. 9A shows Kaplan-Meier survival analysis of mice (n=13-16/group) immunized with PBS control, non-antigen-pulsed cDCs (cDCs), non-antigen-pulsed Th17-inducing DCs (Th17 DCs), antigen-pulsed cDCs (Ag+cDCs) or antigen-pulsed Th17 DCs (Ag+Th17 DCs). ** P<0.01; **** P<0.0001. FIG. 9B shows Kaplan-Meier survival analysis of mice (n=14-25/group) immunized with PBS control or antigen-pulsed Th17 DCs (Ag+Th17 DCs) with or without CD4 (αCD4) or CD8 (αCD8) T cell depletion. FIG. 9C is a graph plotting the mean (+s.e.m., n=6) levels of IL-17 in the ascites fluid of representative tumor-bearing mice at sacrifice in the same groups depicted in FIG. 9A. FIG. 9D includes representative images showing CD3, CD4, and CD8 IHC analysis in tumor tissue harvested at sacrifice in mice treated with PBS, antigen-pulsed cDCs, or antigen-pulsed Th17 DCs. FIGS. 9E-9G are min/max box and whisker plots depicting the levels of CD3 (FIG. 9E), CD4 (FIG. 9F), and CD8 (FIG. 9G) T cells per field analyzed. FIG. 9H includes min-max box and whisker plots showing the levels of antigen-specific antibodies (n=12-18) in blood and ascites. FIG. 9I shows Kaplan-Meier survival analysis of wild-type (WT) or IL-17 knockout (KO) mice (n=5-10/group) immunized with PBS or antigen-pulsed Th17 DCs (Th17 DCs). Inset P values for FIGS. 9A, 9B, and 9I were calculated using Mantel-Cox log rank test. P values for FIGS. 9C-9H were calculated with one-way ANOVA followed by Fisher's LSD test.

FIG. 10 shows that ID8-SP17 tumor cells express MHC class II in vivo. The images show MHC class II staining of peritoneal-derived tumor cells obtained from mice immunized with antigen-loaded cDC or Th17 DC vaccines.

FIG. 11 shows that Th17 DC vaccinated tumor-bearing mice generate durable high avidity antibody responses. The min/max box plot depicts levels of high avidity antigen-specific antibodies (n=12-18) in blood and ascites from moribund mice that succumbed to ovarian cancer. Inset P values (above plots) were calculated with one-way ANOVA followed by Fisher's LSD test.

FIGS. 12A and 12B show that Il17a^{cre} mice do not have a functional IL17A gene. FIG. 12A is a representative image showing PCR products of DNA from Il17a^{cre} (mice 1-5) and B6/J (mice 23-25) using mutant primers. FIG. 12B is a representative image showing PCR products using wild type primers in the same mice.

FIGS. 13A-13F show that Th17 DC vaccination synergizes with immune checkpoint blockade in a CD4 T cell dependent manner. FIG. 13A includes images showing PD-L1 staining of tumor cells derived from the peritoneal cavity on Day 42. FIG. 13B is a Kaplan Meier curve comparing survival in tumor-bearing mice (n=8/group) immunized with PBS, antigen-pulsed Th17 DC vaccines, anti-PD-1 (αPD-1), or the combination of anti-PD-1 and Th17 DC vaccine (*=P<0.05). FIG. 13C is a min/max box and whisker plot (n=9-18) depicting the levels of splenic antigen-specific IFN-γ⁺, IL-4⁺, and IL-17⁺ T cells at Day 42 in tumor-bearing mice immunized and treated with αPD-1 as in FIG. 13B. FIG. 13D is a graph plotting levels of serum antibodies targeting tumor antigens at Day 42. P values in FIGS. 13C and 13D were calculated with one-way ANOVA followed by Fisher's LSD test. FIG. 13E is a min/max box plot of IL-10 levels (pg/ml) in ascites fluid of moribund mice (n=3-10) following treatment as in FIG. 13A. P values were calculated with unpaired Student's T test. FIG. 13F shows a Kaplan Meier curve comparing survival in tumor-bearing mice immunized with antigen-pulsed Th17 DC vaccine and treated with αPD-1 with or without anti-CD4 antibody (n=5-15/group). **=p<0.01. P values for FIGS. 13B and 13F were calculated using Mantel-Cox log rank test.

FIGS. 14A and 14B show that anti-PD-1 does not increase the avidity of Th17 DC vaccine-induced antibodies. FIG. 14A is a min-max box and whisker plot depicting levels of high avidity antigen-specific antibodies (n=12) in Day 42 blood from ovarian cancer-bearing mice treated with PBS, anti-PD-1 (αPD-1), Th17 DC vaccine, or Th17 DC vaccine with anti-PD-1. FIG. 14B is a min-max box and whisker plot depicting % high avidity tumor antigen-specific antibodies (n=12). Inset P values for FIG. 14A were calculated using one-way ANOVA and Fisher's LSD post-hoc test. P values for FIG. 14B were calculated by Mann-Whitney test.

FIGS. 15A-15E show that Th17 DC vaccination alone or in combination results in CD4 T cell driven macrophage and eosinophil infiltration into the peritoneal cavity of tumor-bearing mice. FIG. 15A is a min/max box plot (n=2-7/group) depicting the number of cells recovered from the peritoneal cavity at Day 42 in mice immunized with PBS, Th17 DC vaccine, αPD-1, or a combination of Th17 DC vaccine and αPD-1. NS=not significant, **=p<0.01 by Mann-Whitney Test. FIG. 15B is a min/max box plot depicting the distribution of peritoneal immune cells in the lymphocyte (lymphs), monocyte (DCs/Macs), and granulocyte (Gran) gates from mice treated as in FIG. 15A (n=4-6/group). FIG. 15C is a min/max box plot depicting the relative levels of total and activated (CD69⁺) CD4⁺ and CD8⁺ T cells and B cells and NK cells in the lymphocyte gate at Day 42 following treatment of mice as described in FIG. 15A (n=4-6/group). FIG. 15D is a graph plotting the relative levels of CD11b⁺CD11c⁺ DCs, CD11b⁺F4/80⁺ macrophages (Macs) and CD11b⁺GR-1⁺ myeloid-derived suppressor cells (MDSCs) in the monocyte gate at Day 42 following treatment of mice as described in FIG. 15A (n=4-6/group). FIG. 15E is a graph plotting relative levels of CD11b Ly6G* (neutrophils, Neut), CD11b⁺CD193 SiglecF⁺ (eosinophils, Eosin) and CD11b⁺CD200R3⁺FcεRIα⁺ (Basophils, Baso) in the granulocyte gate at Day 42 following treatment of mice as described in FIG. 15A (n=4-6/group). P values were calculated using a one-way ANOVA followed by Fisher's LSD post-hoc test.

FIGS. 16A-16D show that a subset of patients that developed persistent broad immunity against FRα appeared to be protected against disease recurrence. FIG. 16A is a Kaplan-Meier plot showing recurrence-free survival (RFS) and overall survival (OS) from the time of study enrollment (4-20 weeks after completion of first-line chemotherapy) for all patients eligible for efficacy analysis. FIGS. 16B-16D are graphs plotting IFN-γ, IL-17, and antibody immune response scores (sum of the mean immune responses to the epitopes), respectively, of patients who recurred (squares, n=11) and those who did not recur (circles, n=8). Scores plotted are the mean cumulative T cell or antibody response, by ELIspot or ELISA. T cells are antigen-specific T cells per million PBMC and antibodies are reported in μg/ml.

FIGS. 17A-17C show that the Th17-inducing DC vaccine generated T cell responses in the vast majority of patients. In particular, FIGS. 17A-17C are graphs plotting the percentages of patients who responded to vaccine epitopes with Th1 T cell responses (ELIspot; FIG. 17A), Th17 T cell responses (ELIspot; FIG. 17B), or antibody responses (ELISA; FIG. 17C).

FIG. 18 shows that patients who did not recur had higher ADCC-inducing antibodies. The graph is a scattergram of % dead FRα+ K562 tumor cells coated with patient serum (from phase I) and cultured with ADCC competent NK cells derived from a healthy blood donor. Each dot represents a unique patient. Line=mean and SEM.

FIGS. 19A-19C show a comparison of pre- and post-treatment tumors specimens between patients who relapsed following treatment with Th17 DC vaccine. Specifically, FIGS. 19A-19C are graphs plotting tumor FRα expression, Treg infiltration, and PD-L1 expression, respectively, in primary and corresponding recurrent tumors from n=5-6 patients with recurrence. P values were calculated using the two-sided paired Student's t test. Symbols in some cases overlap, obscuring the number of patients.

FIG. 20 is a Kaplan Meier plot showing that the combination of a Th17 DC vaccination with anti-PD-1 antibody led to long term survival of mice with triple negative breast cancer (TNBC). TNBC-bearing mice were untreated (No Treatment, n=10) or were treated with Th17-DC vaccine (DC Treatment, n=10), anti-PD-1 (PD-1 Treatment, n=10), or a combination of anti-PD-1 and Th17 DC vaccine (DC/PD-1 Treatment, n=8).

### DETAILED DESCRIPTION

Dendritic cells are efficient antigen-presenting cells that express class I and class II major histocompatibility complex (MHC) peptide-presenting molecules on their surfaces, along with a series of costimulatory molecules (Banchereau and Steinman, Nature 392:245-252, 1998). Naïve T cells express receptors for these dendritic cell ligands. Following recognition of peptide-antigen presented in the context of class I or class II molecules, the structure of the T cell membrane is reorganized, bringing together the elements of the T cell receptor with other cell-surface molecules, including the co-receptors CD4 or CD8 and the costimulatory receptors CD28 and CTLA-4 (Monks et al., Nature 395:82-86, 1998; and Wulfing and Davis, Science 282:2266-2269, 1998). Interactions within the newly formed macromolecular complexes determine the outcome of inductive events transduced into T cells by dendritic cells.

This document provides methods and materials for treating mammals having cancer. For example, this document provides methods that include administering, to a mammal having cancer, (a) a Th17 DC vaccine targeted to an antigen such as an FRα polypeptide, and optionally (b) an ICI. Any appropriate mammal can be treated using the methods described herein. For example, humans or other primates such as monkeys can be treated with a Th17 DC vaccine and (optionally) an ICI as described herein. In some cases, dogs, cats, horses, cows, pigs, sheep, mice, or rats can be treated with a Th17 DC vaccine and an ICI as described herein. The mammal to be treated can be identified as having a cancer that is not effectively treatable (or not likely to be effectively treated) with an ICI alone. For example, the mammal can have ovarian cancer, melanoma, chronic lymphocytic leukemia, gastric cancer, cervical cancer, colorectal cancer, breast cancer, or lung cancer.

This document also provides Th17 DC vaccines (e.g., Th17 DC vaccines that can activate IL-17-secreting T cells targeted to an antigen such as an FRα polypeptide), and compositions containing one or more Th17 DC vaccines. Any appropriate method can be used to generate a Th17 DC vaccine. In general, DC vaccines can be generated by isolating DCs or DC precursors from blood or peripheral blood mononuclear cells (PBMCs) obtained from a mammal to be treated. For example, PBMCs can be obtained from whole blood using Ficoll density gradient centrifugation (see, e.g., Disis et al., Clin Cancer Res 5:1289-1297, 1999), and DCs and/or DC precursors (CD14⁺ monocytes) can be isolated from blood or PBMCs by magnetic bead positive selection. The isolated DCs and/or DC precursors can be further matured and activated ex vivo using one or more cytokines, and then incubated with to one or more antigens (e.g., one or more autologous tumor antigens) to generate a Th17 DC vaccine. After maturing and binding to the selected antigen(s), the DC vaccine can be administered to the mammal. Any appropriate agents can be used to promote maturation of DCs such that they activate Th17 cells. For example, to generate Th17 DCs that will activate Th17 cells, DC maturation can be induced by contacting the DCs with IL-15 in combination with an inhibitor of the p38 MAPK pathway (e.g., adezmapimod/SB203580, which is commercially available from Sigma Aldrich, St. Louis, MO). Other agents that can be used in combination with IL-15 to generate Th17 DCs that will activate Th17 cells include, without limitation, methylsulanylimidazole, dormapimod, SB202190, ralimetinib, VX-702, PH-797804, Neflamapimod, and TAK-715. An appropriate number of DCs (e.g., about 2×10⁴ to about 2×10⁶ cells) can be incubated with an appropriate concentration of IL-15 (e.g., about 1 ng/ml to about 100 ng/ml) and an appropriate concentration of p38 MAPK inhibitor (e.g., about 0.15 μM to about 15 μM) for a suitable length of time (e.g., about 1 day to about 7 days).

The matured DCs then can be contacted with one or more antigens. In some cases, the one or more antigens can be peptides derived from a human FRα polypeptide. For example, one or more (e.g., two, three, four, five, six, seven, eight, nine, ten, or more than ten) FRα peptides having any of the amino acid sequences as set forth in TABLE 1 can be incubated with DCs.

TABLE 1

| | SEQ ID | | Length |
|---|---|---|---|
| Sequence | NO: | Positions | (aa) |
| MTTQLLLLLVWVAVVGEAQ | 1 | 5-23 | 19 |
| LLVWVAVVGEAQTRI | 2 | 12-26 | 15 |
| RTELLNVCMNAKHHKEK | 3 | 30-46 | 17 |
| QCRPWRKNACCSTNT | 4 | 56-70 | 15 |
| KDVSYLYRFNWNHCGEMA | 5 | 76-93 | 18 |
| ACKRHFIQDTCLYECS | 6 | 95-110 | 16 |
| LGPWIQQVDQSWRKERV | 7 | 113-129 | 17 |
| VDQSWRKERVLNVPL | 8 | 120-134 | 15 |
| DCEQWWEDCRTSYTCK | 9 | 138-153 | 16 |
| RTSYTCKSNWHKGWNWT | 10 | 147-163 | 17 |
| CKSNWHKGWNWTSGFN | 11 | 152-167 | 16 |
| WHKGWNWTSGFNKCAVGA | 12 | 156-174 | 18 |
| VARFYAAAMSGAGPWA | 13 | 225-240 | 16 |
| PWAAWPFLLSLALMLLWL | 14 | 238-255 | 18 |

In some cases, matured Th17 DCs can be contacted with one or more (e.g., two, three, four, or all five) peptides having any of the amino acid sequences as set forth in SEQ ID NO: 3, SEQ ID NO:4, SEQ ID NO:5, SEQ ID NO:7, and SEQ ID NO:14, to yield Th17 DCs that, when administered to a mammal, can activate Th17 cells against FRα. For example, Th17 DCs can be contacted with a peptide having the sequence set forth in SEQ ID NO: 3, a peptide having the sequence set forth in SEQ ID NO:4, a peptide having the sequence set forth in SEQ ID NO:5, a peptide having the sequence set forth in SEQ ID NO: 7, and a peptide having the sequence set forth in SEQ ID NO:14. It is to be noted, however, that Th17 DCs can be contacted with any suitable tumor antigen, including one or more antigens from HER2, NY-ESO-1, IGFBP-2, hTERT, p53, survivin, or the Mage family of antigens. Moreover, the antigen(s) can be used in any suitable form, including peptide, protein, mRNA, DNA, lipid, or carbohydrate, for example. After incubation for a suitable length of time (e.g., from about 1 hour to about 5 days) with the selected antigens, the Th17 DC vaccine can be administered to the mammal from which the DCs or DC precursors were obtained.

In some cases, in addition to one or more Th17 DC vaccines, the compositions provided herein can include one or more agents (e.g., cytokines) that can promote T cell activation (e.g., IL-2), T cell proliferation (e.g., IL-15), and/or T cell survival (e.g., IL-7). In some cases, a composition provided herein also can contain a pharmaceutically acceptable carrier. Pharmaceutically acceptable carriers include, for example, pharmaceutically acceptable solvents, suspending agents, or any other pharmacologically inert vehicles for delivering a vaccine to a subject.

The methods provided herein include administering a Th17 DC vaccine to mammal by any appropriate route. Administration can be, for example, parenteral (e.g., by intrathecal, intraventricular, intramuscular, intrapleural, or intraperitoneal injection, or by intravenous (i.v.) drip). Administration can be rapid (e.g., by injection) or can occur over a period of time (e.g., by slow infusion). Th17 DC vaccine compositions for parenteral administration can sterile aqueous solutions, which also can contain buffers, diluents, and/or other suitable additives (e.g., penetration enhancers, carrier compounds and other pharmaceutically acceptable carriers).

In some cases, the methods provided herein can include administering one or more ICIs to a mammal. Any appropriate ICI, or combination of ICIs, can be administered to a mammal in the methods provided herein. For example, the methods provided herein can include administering one or more (e.g., one, two, three, four, five, or more than five) ICIs selected from, without limitation, pembrolizumab, atezolizumab, nivolumab, durvalumab, avelumab, tremelimumab, ipilimumab, cemiplimab, and combinations thereof (e.g., ipilimumab in combination with pembrolizumab, atezolizumab, nivolumab, durvalumab, avelumab, tremelimumab, or cemiplimab, or tremelimumab in combination with pembrolizumab, atezolizumab, nivolumab, durvalumab, avelumab, ipilimumab, or cemiplimab). The one or more ICIs can be administered to a mammal by any appropriate route. For example, administration can be parenteral (e.g., by intrathecal, intraventricular, intramuscular, intrapleural, or intraperitoneal injection, or by intravenous (i.v.) drip). Administration can be rapid (e.g., by injection) or can occur over a period of time (e.g., by slow infusion). ICI compositions for parenteral administration can sterile aqueous solutions, which also can contain buffers, diluents, and/or other suitable additives (e.g., penetration enhancers, carrier compounds and other pharmaceutically acceptable carriers).

When a method provided herein includes administering both a Th17 DC vaccine and one or more ICIs to a mammal, the Th17 DC vaccine and the one or more ICIs can be administered to the mammal simultaneously or sequentially, or a combination thereof. In some cases, one or more doses of a Th17 DC vaccine can be administered to the mammal before administration of an ICI. For example, one or more doses (e.g., one, two to four, three to five, four to six, five to seven, six to eight, seven to eight, or more than eight doses) of a Th17 DC vaccine can be administered about one to eight weeks (e.g., one, two, three, four, five, six, seven, eight, one to three, two to four, three to five, four to six, five to seven, or six to eight weeks) before administration of one or more doses (e.g., one, two to four, three to five, four to six, five to seven, six to eight, seven to eight, or more than eight doses) of an ICI. In some cases, administration of a Th17 DC vaccine and an ICI can be initiated at the same time (e.g., on the same day). In some cases, one or more doses (e.g., one, two to four, three to five, four to six, five to seven, six to eight, seven to eight, or more than eight doses) of a Th17 DC vaccine can be administered, and then one or more doses (e.g., one, two to four, three to five, four to six, five to seven, six to eight, seven to eight, or more than eight doses) an ICI can be administered along with one or more additional doses (e.g., one, two to four, three to five, four to six, five to seven, six to eight, seven to eight, or more than eight additional doses) of the Th17 DC vaccine.

Methods for treating a mammal (e.g., a human) having cancer (e.g., OC) can include administering, to the mammal, an effective amount of a Th17 DC vaccine and, optionally, an effective amount of an ICI. In some cases, an effective amount of a Th17 DC vaccine and an effective amount of an ICI can be amounts that, in combination, reduce one or more symptoms associated with a cancer within a mammal, reduce the number of tumor cells within a mammal, reduce the size of a tumor within a mammal, or prolong progression free survival, recurrence free survival, and/or overall survival of the mammal, without producing significant toxicity to the mammal. In some cases, an effective amount of a Th17 DC vaccine containing Th17 DCs can be an amount that contains from about 5×10⁵ Th17 DCs to about 5×10⁸ Th17 DCs (e.g., from about 5×10⁵ Th17 DCs to about 5×10⁶ Th17 DCs, from about 5×10⁶ Th17 DCs to about 5×10⁷ Th17 DCs, or from about 5×10⁷ Th17 DCs to about 5×10⁸ Th17 DCs). An effective amount of an ICI can be from about 50 mg to about 1500 mg (e.g., from about 50 mg to about 100 mg, from about 100 mg to about 250 mg, from about 250 mg to about 500 mg, from about 500 mg to about 750 mg, from about 750 mg to about 1000 mg, from about 1000 mg to about 1250 mg, or from about 1250 mg to about 1500 mg). The effective amount can remain constant or can be adjusted as a sliding scale or variable dose depending on the mammal's response to treatment. Various factors can influence the actual effective amount used for a particular application. For example, the severity of the cancer when treating a mammal having such a disease, the route of administration, the age and general health condition of the mammal, excipient usage, the possibility of co-usage with other therapeutic treatments such as use of other anti-cancer agents (e.g., chemotherapy drugs), and the judgment of the treating physician may require an increase or decrease in the actual effective amount of a Th17 DC vaccine and/or an ICI that is administered. After treatment, the mammal can be monitored for both responsiveness to the treatment and toxicity symptoms. If a particular mammal fails to respond to a particular amount, then the amount of Th17 DC vaccine and/or ICI administered can be increased by, for example, two fold. After receiving the higher amount(s), the mammal can be further monitored for both responsiveness to the treatment and toxicity symptoms, and further adjustments can be made accordingly.

In some cases, an effective frequency of administration of a Th17 DC vaccine described herein and, optionally, an ICI can be a frequency that reduces one or more symptoms associated with a cancer in the mammal, reduces the number of tumor cells within the mammal, reduces the size of a tumor within the mammal, or prolongs progression free survival, recurrence free survival, and/or overall survival of the mammal, without producing significant toxicity to the mammal. In some cases, an effective frequency of administration of a Th17 DC vaccine described herein and (optionally) an ICI can be a frequency that reduces one or more symptoms associated with a cancer in a mammal as compared to the mammal prior to treatment. For example, an effective frequency of administration of a Th17 DC vaccine described herein and (optionally) an ICI (e.g., a Th17 DC vaccine targeted to FRα and pembrolizumab) can be, independently or in combination, from about three times a week to about once a month (e.g., twice a week, once a week, once every 14 days, once every 21 days, or once every 28 days). It is to be noted that the effective frequency of administration of the Th17 DC vaccine is not necessarily the same as the effective frequency of administration of the ICI. The frequency of administration of a Th17 DC vaccine described herein and (optionally) an ICI can remain constant or can be variable during the duration of treatment. Various factors can influence the actual effective frequency used for a particular application. For example, the effective amount, the severity of the cancer when treating a mammal having such a cancer, the route of administration, the age and general health condition of the mammal, excipient usage, the possibility of co-usage with other therapeutic or prophylactic treatments such as use of other anti-cancer agents (e.g., chemotherapy drugs), and the judgment of the treating physician may require an increase or decrease in the actual effective frequency of administration of a Th17 DC vaccine provided herein and, optionally, an ICI.

In some cases, an effective duration of administration of a Th17 DC vaccine described herein and, optionally, an ICI can be a duration that reduces one or more symptoms associated with a cancer in a mammal, reduces the number of tumor cells within a mammal, reduces the size of a tumor within a mammal, or prolongs progression free survival, recurrence free survival, and/or overall survival of the mammal, without producing significant toxicity to the mammal. In some cases, an effective duration of administration of a Th17 DC vaccine described herein and (optionally) an ICI can be a duration that reduces one or more symptoms associated with a cancer in a mammal having such cancer as compared to the mammal prior to treatment. For example, an effective duration of administration of a Th17 DC vaccine provided herein and (optionally) an ICI (e.g., a Th17 DC vaccine targeted to FRα and pembrolizumab), independently or in combination, can vary from a single time point of administration to administration over the course of several weeks to several months (e.g., 2 to 4 weeks, 4 to 8 weeks, 8 to 12 weeks, 12 to 16 weeks, or more than 16 weeks). It is to be noted that the effective duration of administration of a Th17 DC vaccine is not necessarily the same as the effective duration of administration of an ICI. Multiple factors can influence the actual effective duration used for a particular application. For example, the severity of the cancer, the effective frequency, the effective amount, the route of administration, the age and general health condition of the mammal, excipient usage, the possibility of co-usage with other therapeutic or prophylactic treatments such as use of other anti-cancer agents (e.g., chemotherapeutic agents), and the judgment of the treating physician may require an increase or decrease in the actual effective duration of administration of a Th17 DC vaccine provided herein and, optionally, an ICI. In some cases, for example, a Th17 DC vaccine can be administered once every 3 to 6 months for about 1 to 2 years, and an ICI can be administered once every 2 to 6 weeks for about 1 to 5 years.

In some cases, the methods provided herein can include monitoring a mammal after treatment with a Th17 DC vaccine, or after treatment with a Th17 DC vaccine and an ICI, to assess the effectiveness of the treatment. In some cases, for example, a course of treatment and/or the severity of one or more symptoms related to the cancer being treated can be monitored. Any appropriate method can be used to determine whether or not a mammal having cancer is responding to treatment. For example, clinical scanning techniques (e.g., computed tomography (CT), positron emission tomography (PET)/CT, bone scan, and magnetic resonance imaging (MRI)) can be used to assess the presence, absence, or physical characteristics (e.g., size) of a cancer within a mammal (e.g., a human) treated by the methods provided herein. In some cases, the effectiveness of treatment with a Th17 DC vaccine and, optionally, an ICI can be assessed based on the length of RFS, PFS, or OS, as compared to an average RFS, PFS, or OS of a mammal with the same type of cancer that was not treated with the Th17 DC vaccine and, optionally, the ICI.

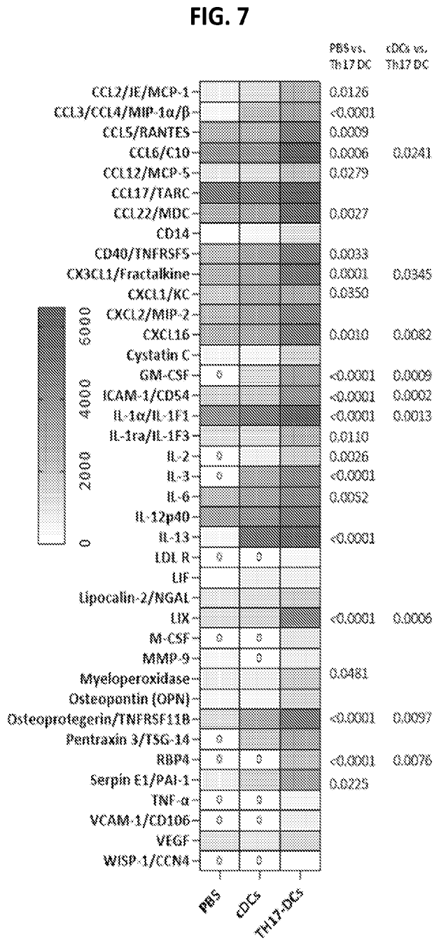

In some cases, the methods provided herein can include monitoring a mammal after treatment for expression of one or more markers that can indicate interaction of Th17 DC vaccine-elicited T cells with antigen-presenting cells. For example, as described herein, administration of a Th17 DC vaccine induced CD4 T cells that stimulated elevated levels of myeloid cell modulating cytokines/chemokines, including CCL6, CX3CL1, CXCL16, GM-CSF, ICAM-1, IL-1a, LIX, osteoprotegerin, and RBP-4 (see, FIG. 7). Thus, in some cases, the methods provided herein can include monitoring a mammal after treatment for elevated expression of one or more cytokines/chemokines such as, without limitation CCL6, CX3CL1, CXCL16, GM-CSF, ICAM-1, IL-1a, LIX, osteoprotegerin, and RBP-4, where elevated expression of the one or more cytokines/chemokines indicates effective treatment. As used herein, an “elevated” level of a marker refers to a level of the marker (either mRNA or protein) that is higher than the level of the marker in the mammal prior to administration of a Th17 DC vaccine. For example, the level of a cytokine/chemokine mRNA or protein (e.g., one or more of CCL6, CX3CL1, CXCL16, GM-CSF, ICAM-1, IL-1a, LIX, osteoprotegerin, and RBP-4) in a sample (e.g., a blood sample) can be considered to be “increased” if the level is at least 5% (e.g., at least 10%, at least 15%, at least 20%, at least 25%, at least 30%, at least 40%, at least 50%, at least 75%, or more than 100%) greater than the level in a corresponding sample from the mammal prior to administration of a Th17 DC vaccine.

### EXEMPLARY EMBODIMENTS

Embodiment 1 is a method for treating a mammal having cancer, wherein the method comprises administering, to the mammal, an effective amount of a vaccine composition capable of activating T cells within the mammal to form activated IL-17-secreting T cells that can recognize one or more antigens expressed by said cancer.

Embodiment 2 is the method of embodiment 1, wherein the mammal is a human.

Embodiment 3 is the method of embodiment 1 or embodiment 2, wherein the cancer is ovarian cancer, melanoma, chronic lymphocytic leukemia, gastric cancer, cervical cancer, colorectal cancer, breast cancer, or lung cancer.

Embodiment 4 is the method of any one of embodiments 1 to 3, wherein the vaccine composition comprises dendritic cells (DCs) presenting one or more folate receptor alpha (FRα) antigens.

Embodiment 5 is the method of embodiment 4, wherein the one or more FRα antigens comprise one or more peptides having the amino acid sequence set forth in SEQ ID NO: 3, SEQ ID NO:4, SEQ ID NO:5, SEQ ID NO:7, or SEQ ID NO:14.

Embodiment 6 is the method of any one of embodiments 1 to 5, wherein the method comprises administering a dose of about 5×10⁶ to about 50×10⁶ of the DCs composition to the mammal.

Embodiment 7 is the method of any one of embodiments 1 to 6, wherein the method comprises administering the vaccine composition to the mammal two or more times over a time period of about 2 weeks to about 16 weeks.

Embodiment 8 is the method of any one of embodiments 1 to 7, wherein the method further comprises monitoring the mammal after the administering step to determine an effect of the vaccine composition on the mammal.

Embodiment 9 is the method of embodiment 8, wherein the monitoring comprises using computed tomography, positron emission tomography/computed tomography, bone scan, or magnetic resonance imaging.

Embodiment 10 is the method of embodiment 8, wherein the monitoring comprises measuring the level of one or more biomarkers in a tumor biopsy obtained from the mammal after the administering step, wherein the one or more biomarkers are selected from the group consisting of CCL6, CX3CL1, CXCL16, GM-CSF, ICAM-1, IL-1a, LIX, osteoprotegerin, and RBP-4.

Embodiment 11 is the method of any one of embodiments 1 to 10, further comprising administering, to the mammal, an effective amount of an immune checkpoint inhibitor (ICI).

Embodiment 12 is the method of embodiment 11, wherein the ICI is selected from the group consisting of pembrolizumab, atezolizumab, nivolumab, durvalumab, avelumab, tremelimumab, ipilimumab, and cemiplimab.

Embodiment 13 is the method of embodiment 11 or embodiment 12, wherein the method comprises administering a dose of about 50 mg to about 1500 mg of the ICI to the mammal.

Embodiment 14 is the method of any one of embodiments 11 to 13, wherein the method comprises administering the ICI to the mammal two or more times over a time period of about 2 weeks to about 16 weeks.

Embodiment 15 is the method of any one of embodiments 11 to 14, wherein the method further comprises monitoring the mammal after the administering steps to determine an effect of the vaccine composition and the ICI on the mammal.

Embodiment 16 is the method of embodiment 15, wherein the monitoring comprises using computed tomography, positron emission tomography/computed tomography, bone scan, or magnetic resonance imaging.

Embodiment 17 is the method of embodiment 15, wherein the monitoring comprises measuring the level of one or more biomarkers in a tumor biopsy obtained from the mammal after the administering steps, wherein the one or more biomarkers are selected from the group consisting of CCL6, CX3CL1, CXCL16, GM-CSF, ICAM-1, IL-1a, LIX, osteoprotegerin, and RBP-4.

The invention will be further described in the following examples, which do not limit the scope of the invention described in the embodiments.

### EXAMPLES

### Example 1-Th17 T Cell-Inducing Vaccines Subvert Resistance of Ovarian Cancer to Immune Checkpoint Blockade

### Materials and Methods

Generation of DC vaccines: Bone marrow cells were obtained from C57BL/6J mice as described elsewhere (Lamichhane et al., Cancer Res 77:6667-6678, 2017). Cells were resuspended at 0.2×10⁶/mL with RPMI with 10% FBS media containing murine GM-CSF (50 ng/mL, R&D Systems, Minneapolis, MN, Cat. #415-ML-050/CF) and IL-4 (20 ng/mL, R&D Systems, Cat. #404-ML-100/CF) with (Th17 DC vaccines) or without (conventional DC vaccines; cDCs) IL-15 (10 ng/ml, R&D systems, #447-ML-010/CF) and p38 MAPK inhibitor SB203580 (1.5 M, Sigma Aldrich, St. Louis, MO, Cat. #S8307). A half volume of fresh media was added on Day 4 and 6. Tumor cell lysates from ID8 ovarian cancer cells (1:10 dilution) were pulsed on the DCs on Day 7, followed by stimulation with LPS (200 μg/mL) for 16 hours. The cells were removed from the plates by gentle scraping, washed with PBS twice, and resuspended at 10⁷ cells per ml before use as vaccines.

p38 MAP kinase assay: Dendritic cells treated with and without SB203580 (1.5 μM, Sigma Aldrich) were rinsed with cold PBS, scraped in 0.5 mL cold 1× cell lysis buffer (Cell Signaling Technologies, Danvers, MA, Cat. #9803) plus 1 mM phenylmethylsulfonyl fluoride (PMSF) and sonicated. Beads containing immobilized antibody (phospho-p38 MAPK (Thr180/Tyr182)) were incubated with the DC cell lysates overnight with gentle rocking at 4° C. The beads were then centrifuged at 14,000×g for 30 seconds at 4° C., washed twice with 500 μL of 1× cell lysis buffer, and washed twice with 500 μL of 1× kinase buffer (Cell Signaling Technologies, Cat. #9802), all while on ice. The pellet was resuspended in 50 μL of 1× kinase buffer supplemented with 200 μM ATP, and kinase substrate (ATF-2 fusion protein) was added and incubated for 30 minutes at 30° C. The reaction was terminated with 25 μL 3×SDS sample buffer, vortexed, and centrifuged for 30 seconds at 14,000×g. The sample was heated at 95° C. for 2-5 minutes followed by standard immunoblotting probing for phospho-ATF-2 (Thr71) as described below.

Immunoblotting: Cells were harvested and lysed using 1× cell lysis buffer (Cell Signaling Technologies). Samples were sonicated and BioRad protein assays were performed to quantify the amount of protein in each sample. Proteins were denatured for 10 minutes at 70° C., and 30 μg of protein were loaded onto SDS-PAGE gels. Proteins were transferred to PVDF membranes using iBlot and the membranes were blocked for 1 hour at room temperature using Li-Cor Odyssey buffer (Li-Cor Biosciences, Lincoln, NE, Cat. #927-40000). Primary antibody (anti-phospho-ATF-2 (Thr271), Cell Signaling Technologies, Cat. #9221, or β-Actin, Li-Cor Bioscience, Cat. #926-42212) was added (1:500-1:1000 dilution) and incubated either overnight at 4° C. or for 2 hours at RT. Membranes were then washed three times with 1×PBST, and incubated with HRP-conjugated secondary antibody against IgG for 1 hour at RT. Membranes were washed and developed using Odyssey.

Flow cytometry: Staining of cell surface immune markers was performed on ascites and peritoneal cavity cells. Cells were resuspended at 1×10⁶ cells per 100 μL in a U-bottom plate, spun down directly in the U-bottom plate, and incubated with Fixable viability stain for 20 minutes. Cells were washed with PBS and incubated with CD16/32 blocking antibody (Miltenyi Biotech, Cat. #130-092-574) for 10 minutes at 4° C., and cell surface antibodies were added to the samples. Samples were run on an Attune flow cytometer (Invitrogen, Waltham, MA) and data analysis was performed using Flow jo software (FlowJo 10.6.2). Similar numbers of events, usually 200,000 to 400,000, were collected for all groups. Appropriate FMO were used as controls. Antibodies used were as follows: anti-CD3 (Invitrogen, clone 145-2C11, Cat. #17-0031-82), anti-CD4 (BD Pharmingen, San Diego, CA, clone GK1.5, Cat. #552051), anti-CD8 (BioLegend, San Diego, CA, clone 53.6.7, Cat. #100748), anti-CD69 (BioLegend, clone H1.2F3, Cat. #104530), anti-GR1 (BioLegend, clone RB6-8C5, Cat. #108456), anti-CD11b (BioLegend, clone M1-70, Cat. #101263), anti-CD19 (BioLegend, clone 6D5, Cat. #115546), anti-CD11c (Invitrogen, clone N418, Cat. #17-0114-82), anti-F4/80 (Bio-Rad, Hercules, CA, clone A3-1, Cat. #MCA497A700), anti-CD45 (BioLegend, clone 30-F11, Cat. #103128), anti-CD80 (BioLegend, clone 16-10A1, Cat. #104706), anti-CD86 (BioLegend, clone GL-1, Cat. #105024), anti-OX40L (BioLegend, clone RM134, Cat. #108812), anti-MHCI (BioLegend, clone AF6-88.5, Cat. #116502), anti-MHCII (BioLegend, clone M5/114.15.2, Cat. #107632), anti-CD193 (BioLegend, clone J073E5 Cat. #144510), anti-Siglec-F (BioLegend, clone S17007L, Cat. #155509), anti-Ly-6G (Tonbo Biosciences, San Diego, CA, clone 1A8. Cat. #85-1276-U100), anti-CD200R3 (BioLegend, clone Ba13, Cat. #142207), anti-FceR1a (BioLegend, clone MAR-1, Cat. #134305) and anti-NK1.1 (BioLegend, clone, PK136, Cat. #108732).

Cytokine ELISAs: ELISA kits were used to detect IL-10 (Cat. #88-7105-86), IL-17A (Cat. #88-7371-86), IL-12p70 (Cat. #88-7121-88), IL-6 (Cat. #88-7064-88), IL-1B (ThermoFisher scientific, Cat. #88-7013-88), TGF-β (Cat. #88-8350-88), and IFN-γ (Cat. #88-7314-88). All kits were purchased from ThermoFisher Scientific (Waltham, MA). 96-well plates were coated overnight with coating antibody diluted in coating buffer. The plates were washed three times using an AquaMax 4000 (Molecular Devices, San Jose, CA) plate washer and blocked with 200 μL of ELISA diluent (ThermoFisher Scientific, Cat. #00-4202-56) for an hour. Plates were washed and samples were added to the standards. Culture media samples were added without any dilution, while serum from blood or ascites was added at 1:10 dilution, followed by overnight incubation.

Plates were washed and detecting antibody diluted in ELISA buffer was added and incubated for one hour. Plates were washed and avidin/streptavidin was added for 30 minutes, followed by washing and developing with TMB substrate. Reactions were stopped using IN HCL. Absorbance was measured at 450 nm. Values were subsequently converted into a cytokine concentration using a standard curve.

Antibody ELISAs and Urea Treatment: Flat-bottom polystyrene 96-well plates were coated overnight at 4° C. with 100 μL/well of 1×PBS containing 10 μg/mL of ID8 tumor cell lysate. Mouse IgG (Sigma, St. Louis, MO) was used as a protein standard that was added at a concentration range of 0.195 to 1000 ng/well. All wash steps were carried out with PBS containing 0.05% TWEEN® 20 using an AquaMax 4000 plate washer. PBS containing 1% BSA was used as blocking and assay buffer. The wells were blocked with 200 L/well of the blocking/assay buffer and incubated for 1 hour at RT on a rocking platform. After washing again, mouse serum from control or treated mice was added to the plate at a 1:25 dilution in triplicate wells and incubated at 37° C. for 1 hour. The plates were washed, and one set of mouse serum was treated with 200 μL of 6M urea (Sigma Aldrich, Cat #U5378-100g) while the other set of mouse serum was treated with 200 μL of wash buffer. The plates were incubated for 30 minutes at 37° C. with rigorous shaking. After washing, 100 μL/well of goat anti-mouse IgG HRP (Santa Cruz Biotechnology, Dallas TX) was diluted 1:2,000 and incubated on the wells for 1 hour at 37° C. After a final wash, each well was incubated with 100 μL TMB substrate (BD Biosciences, Mississauga, ON, Canada). Color development was stopped by the addition of 50 μL/well of diluted HCL. Absorbance was read at 450 nm on a plate reader. Wells that were not coated with the tumor cell lysate were used to subtract the background from the mouse serum samples. These values were subsequently converted into an antibody concentration using an IgG standard curve generated in the same assay.

IL-4, IFN-γ, and IL-17 ELIspots: Spleen cells (0.5×10⁶ cells) from C57BL/6J mice were incubated at 37° C. with and without tumor cell lysate in a 96-well U-bottom plate on Day 1. For IFN-γ (MabTech, Cincinnati, OH, cat #3321-2H) and IL-4 (MabTech, cat #3311-2H) ELISpot, plates (MSIP4510, MilliporeSigma, Burlington, MA) were treated with 35% ethanol for 1 minute followed by 5 washes with water. Anti-IFN-γ (clone AN18) or anti-IL-4 (clone 11B11) coating antibody purchased from Mab Tech was diluted and coated on the plates, which were sealed and incubated at 4° C. overnight. After 24 hours, the plates were blocked with 200 μL of media for 1 hour. Cells were transferred from the U-bottom plate to the ELIspot plate and incubated at 37° C. overnight. Plates were washed and incubated with biotinylated anti-IFN-γ antibody (clone #R4-6A2-Biotin, MabTech) or anti-IL-4 (clone #BVD6-24G2-Biotin, Mab Tech) for 1 hour followed by avidin-conjugated HRP for 30 minutes. Plates were washed and developed using 3-amino-9-ethylcarbazole (AEC) substrate until clear spots were seen. For IL-17 ELIspots, precoated 96-well plated were obtained from R&D Systems (cat #EL421) were blocked with 200 μL of culture media for 20 minutes at room temperature. Media were aspirated and the cells were plated along with stimulants and incubated at 37° C. for 48 hours, washed, and then incubated with 100 μL of detection antibody overnight at 4° C. Plates were washed and 100 μL of diluted streptavidin-conjugated alkaline phosphatase were added to each well, followed by incubation at RT for 2 hours. Plates were washed and 100 μL of BCIP/NBT chromogen was added into each well and incubated for 1 hour in the dark, followed by washing with water. All plates were dried overnight, and spots were read the next day using Advanced imaging Devices (AID) ELISpot software (Version 7, AID GmbH, Germany) ELISpot reader.

Immunohistochemistry on ascites and peritoneal cavity cells: Ascites obtained from ovarian tumor-bearing mice C57BL/6J mice were gently layered on top of a discontinuous Ficoll gradient consisting of a lower 100% layer and an upper 75% layer. The ascites and Ficoll gradient were centrifuged at 280×g for 30-45 minutes at 4° C., followed by two washes in Hank's balanced salt solution. Tumor infiltrating lymphocytes (TILs) were collected from the top layer. To obtain peritoneal cavity cells, the mice were euthanized and 20 mL of PBS were injected intraperitoneally to wash the peritoneum, which was drained into a centrifuge tube. Both ascites and peritoneal cells were washed in PBS and subjected to ammonium-chloride-potassium (ACK) treatment for 1 minute at RT. The ACK was diluted in PBS and the cells were centrifuged at 300×g for 5 minutes and the cell numbers were calculated. The cells were fixed in formalin (4% formaldehyde) for 20 minutes at room temperature and washed 2-3 times with PBS. After removing the supernatant, 500 μL of Epredia Histogel (Fisher Scientific, Waltham, MA, cat. #HG-4000-012) was added to the cell pellet, which was then air dried for 15 minutes. The gel was transferred to a cassette and was sent to the Mayo Clinic histochemistry core for staining. Cells were stained for Ki67 (Abcam, Cambridge, UK, cat. #15580), Sp17 (Proteintech, Rosemont IL, cat. #13367-1-AP), MHC-II (Thermo Fisher Scientific, cat #14-5321-82), PD-L1 (Cell Signaling Technology, cat #13684), CD3 (Abcam, cat. #ab166689), CD4 (Abcam, cat. #ab183685) and CD8 (Invitrogen, cat. #ab14-0808-82). The stained sections were scanned using an AT2 slide scanner (Leica Biosystems, Inc., Buffalo Grove, IL) and analyzed using eSlide Manager Software (Leica Biosystems, version #v.12.4.3.5008).

T cell co-culture and Proteome Profiler Mouse XL Cytokine Array: T cells primed in vivo with the vaccine were simulated ex vivo with cDCs pulsed with the antigen. Briefly, mice were inoculated with tumor and subsequently vaccinated for 4 weeks and rested for 2 weeks before harvest. Spleens, peripheral blood, and peritoneal cavity cells were harvested from these mice. Spleen cells were processed for single cell suspensions and CD4+ T cells were removed with a CD4+ isolation kit (Miltenyi Biotech) using AutoMacs and co-cultured with cDCs pulsed with antigen at a 1:1 ratio in 1 mL media in a 12 well plate for 72 hours. Media were harvested and centrifuged, and supernatants were used to determine which cytokines were secreted by different groups of vaccinated mice. The proteome profile mouse XL cytokine array kit (R&D Systems, cat. #ARY028) was used to test the cytokines that were secreted in the co-culture experiment, following the manufacturer's instructions.

Animals: Six to eight week-old female C57BL/6J (B/6J) and STOCK IL17a^{tm1.1(icre)Stck}/J (Il17a^{cre}) mice were used for the experiments described herein. Both strains were purchased from the Jackson Laboratory (Bar Harbor, ME), and Il17a^{cre} was maintained as a colony.

Ovarian cancer cell line and tumor challenge: ID8 tumor cells were obtained from Dr. K. Roby (University of Kansas; Lawrence, KS) and were authenticated by IDEXX Bioanalytics (Columbia, MO). The ID8 cell line was transfected with SP17 for biomarker purposes. SP17 is a cancer-testis antigen that is overexpressed in ovarian cancer (Brunette et al., BMC Cancer 18:970, 2018). The SP17 gene was cloned into the pCDH lentiviral vector, which was transfected into ID8 cells with psPAX2 and pMD2.G packaging vectors. Cells were screened using puromycin, and expression of the SP17 gene was confirmed by Western blotting. To establish peritoneal carcinomatosis, ID8-SP17 tumor cells (4×10⁶ cells) were injected i.p. in a volume of 400 μL PBS. Blood, ascites, spleen, and omentum were harvested from the mice when moribund, usually 50 to 320 days following challenge. Peritoneal cavity cells were obtained at day 42 following tumor challenge and treatment using PBS flushing as described above.

Tumor cell lysate preparation: ID8-SP17 cell lines were grown in DMEM supplemented with 10% FBS, and 1 mg/mL puromycin was added for selection. Cells were harvested and resuspended at 10⁷ cells/mL in PBS. Cells were subjected to five cycles of freezing on dry ice for 30-60 minutes followed by thawing at 37° C. The supernatants were collected and protein concentrations were measured using the BSA assay. When the tumor lysates were used, 70 to 100 mg/mL of protein typically were added to the DC cultures from bone marrow on Day 7 at a 1:10 dilution.

Vaccination and immune checkpoint blockade therapy: Dendritic cell vaccines (DC vaccines) were harvested from bone marrow cultures (described above) and washed twice in PBS, and the cells were counted and adjusted to a final concentration of 10⁷ cells/mL in PBS. 100 μL of the cells were injected into mice subcutaneously (s.c.) or intraperitoneally (i.p.). 200 mg hamster IgG (Jackson ImmunoResearch, Westgrove PA) or 200 mg G4 clone PD-1-blocking monoclonal antibody were given i.p twice a week for 10 weeks after 13-15 days of tumor inoculation (Lamichhane et al., supra; and Karyampudi et al., Cancer Res 74:2974-85, 2014). For in vivo T cell depletion studies, anti-CD4 antibody (GK1.5) or anti-CD8 antibody (53.6.7) was given at 0.5 mg/dose i.p. in parallel with the vaccines on a weekly basis for four weeks starting one week after tumor inoculation. Anti-PD-1 and antibodies for depletion were provided by the Mayo Clinic Antibody Hybridoma Core.

Phagocytosis assays: ID8-Sp17 tumor cells were labelled with PKH26 Red fluorescent cell linker per the manufacturer's instructions (Sigma Aldrich, cat. #PKH26GL). These cells were used to generate tumor cell lysates as described above. Dendritic cells were co-cultured with these labelled tumor cell lysates at a cell ratio of 1:1 for 24 hours at 37° C., followed by stimulation with LPS for 16 hours. Cells were harvested and stained for dendritic cell markers, and the uptake was measured by flow cytometry.

Genotyping: IL-17a^{cre} mice were genotyped at the IL-17a locus with standard polymerase chain reaction (PCR) using the mutant reverse B primer ACTCCCTCACATCCTCAGGTT (SEQ ID NO:15), the wild type reverse A primer CTTAGTGGGTTAGTTTCACAGC (SEQ ID NO:16), and the common A, B primer CAAGTGCACCCAGCACCAGCTGATC (SEQ ID NO:17). The expected result for IL17a^{cre} was a 300 bp product, and the expected result for B/6J was a 304 bp product.

Statistical analysis: All statistical tests were two sided with p value <0.05 being considered statistically significant. Statistical tests were calculated using GraphPad Prism V.8.

### Results

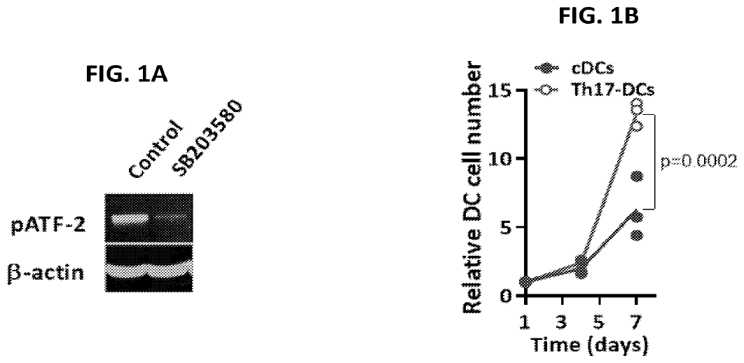

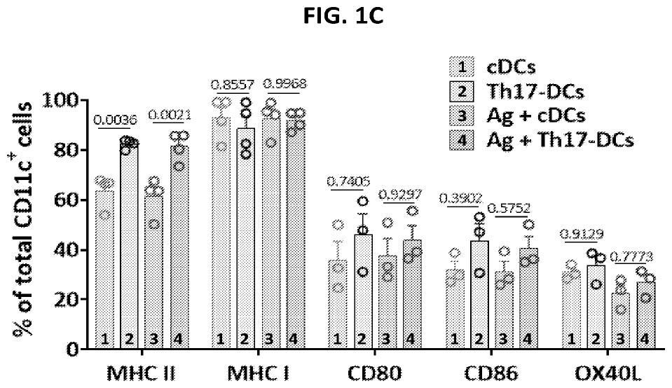

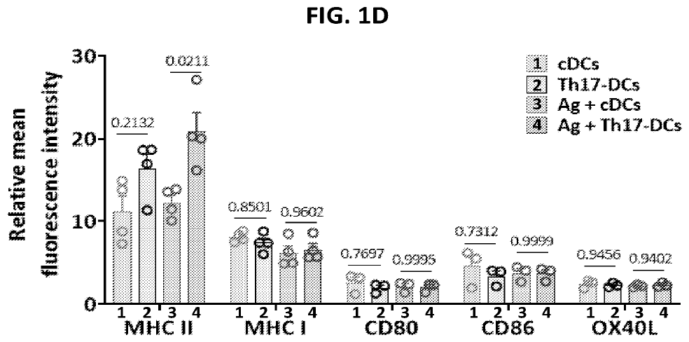

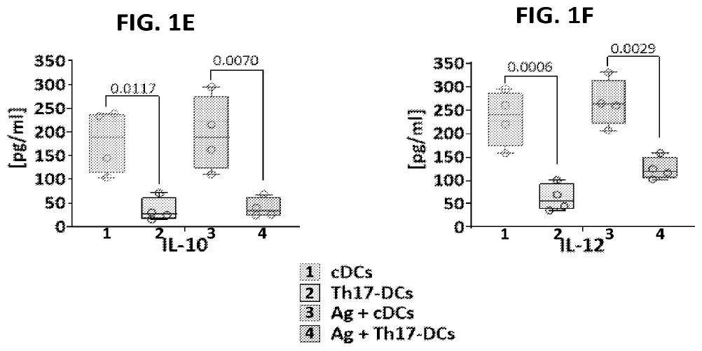

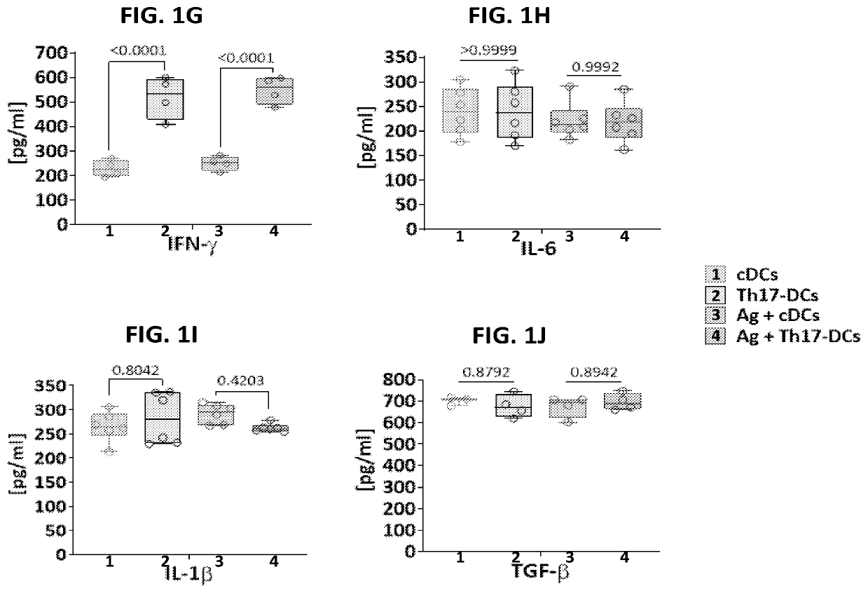

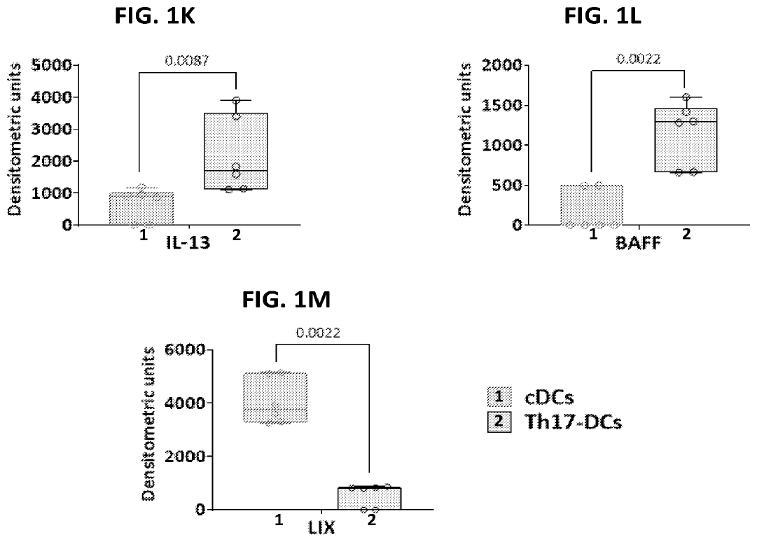

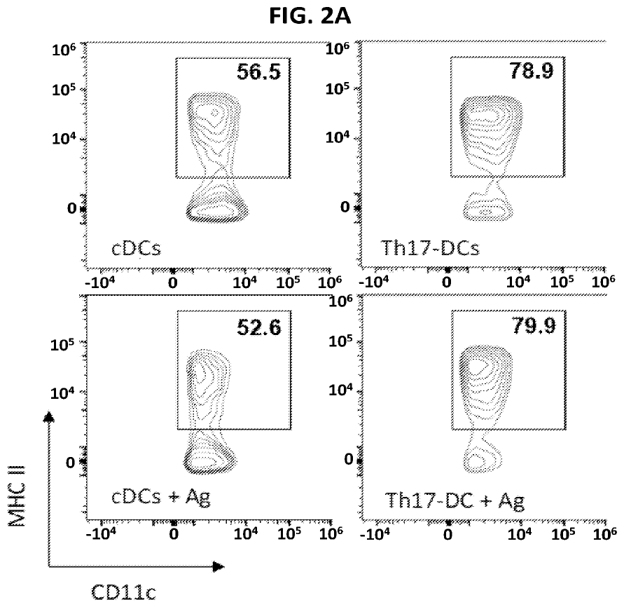

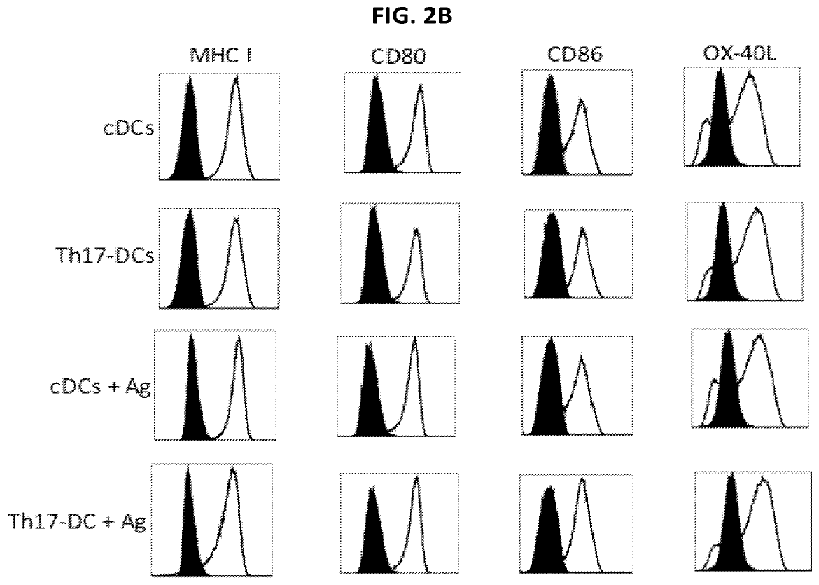

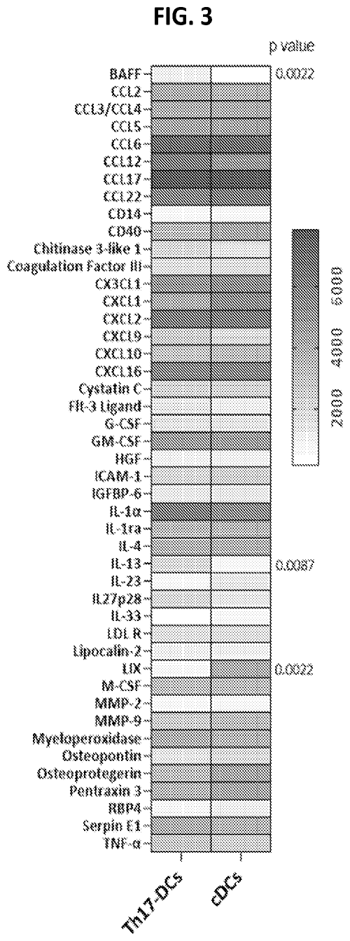

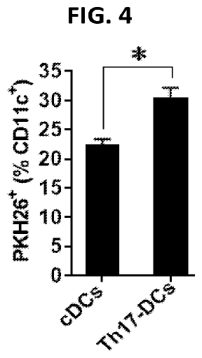

IL-15 costimulation and p38 MAPK blockade selectively upregulates expression of MHC class II and IFN-γ while suppressing IL-10 and IL-12 production: SBA203580 (adezmapimod) was used to block p38 MAPK activity in DCs. SBA203580 is a selective inhibitor of p38 MAPK isoforms, with little activity against other MAPKs such as p44/p42 MAPK or SAPK/JNK (Kumar et al., Biochem Biophys Res Commun 263:825-831, 1999). Initial studies focused on verification of effectiveness in murine DCs demonstrating nearly complete abolition of phosphorylation of p38 MAPK target, activating transcription factor 2 (ATF-2) with little toxicity and improved recovery of DCs relative to conventional DC methods (FIGS. 1A and 1B) (Sreekanth et al., PLOS One 11: e0149486, 2016). The addition of p38 MAPK inhibitor and IL-15 selectively upregulated both the numbers of MHC class II⁺ DCs and the magnitude of MHC class II expression (FIGS. 1C, 1D, and 2A). The impact on cell surface expression of T cell activators was highly specific, as there were no significant increases in MHC I, CD80, CD86, or OX40L expression (FIGS. 1C, 1D, and 2B). Exposure to tumor antigen had no impact overall on expression of MHC class II or the other T cell activators (FIGS. 1C, 1D, 2A, and 2B). Similarly, exposure of DCs to IL-15 and p38 MAPK inhibitor demonstrated a highly selective cytokine production response. Release of both IL-10 (a Treg inducer) and IL-12 (a Th17 inhibitor) was markedly suppressed, while IFN-γ release was elevated (FIGS. 1E-1G). Levels of IL-6, IL-1β, and TGF-β, which are known inducers of Th17 T cell responses (Harbour et al., Sci Immunol 5 (49): eaaw2262, 2020; Chung et al., Immunity 30:576-587, 2009; and Mangan et al., Nature 441:231-234, 2006), were maintained at high levels by the DCs and were not impacted by IL-15 or p38 MAPK inhibitor (FIGS. 1H-1J). Again, exposure to antigen had little impact on cytokine release. Additional qualitative proteomic analysis of myeloid-produced cytokines showed extensive similarity between conventional DC maturation and DCs matured in the presence of p38-MAPK inhibitor and IL-15 co-stimulation, with notable exceptions (FIG. 3 and TABLE 1). Specifically, in the presence of the p38-MAPK inhibitor and IL-15, the B cell regulators BAFF and IL-13 were both significantly elevated (FIGS. 1K and 1L). In contrast, the Treg chemoattractant LIX (CXCL5) was suppressed (FIG. 1M) (Shi et al., Eur J Immunol 44:420-430, 2014). In addition, blockade of p38 MAPK and co-stimulation with IL-15 modestly increased phagocytosis (FIG. 4). Thus, the combination of IL-15 and p38-MAPk inhibitor induces a phenotype of DCs that favors activation of Th17 T cells and B cells rather than Tregs.

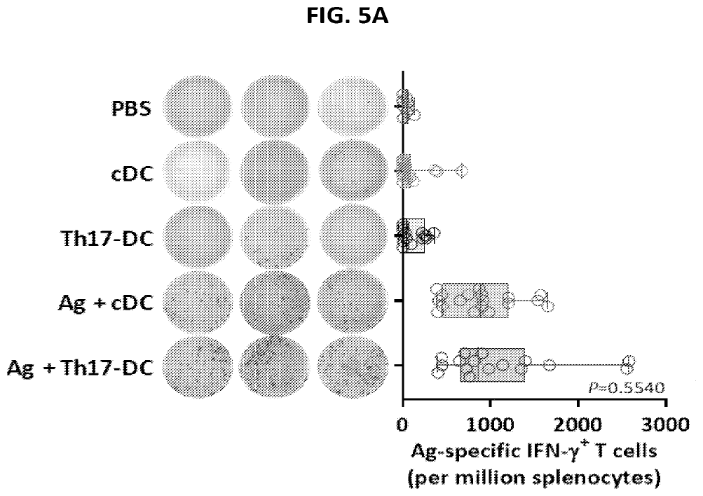

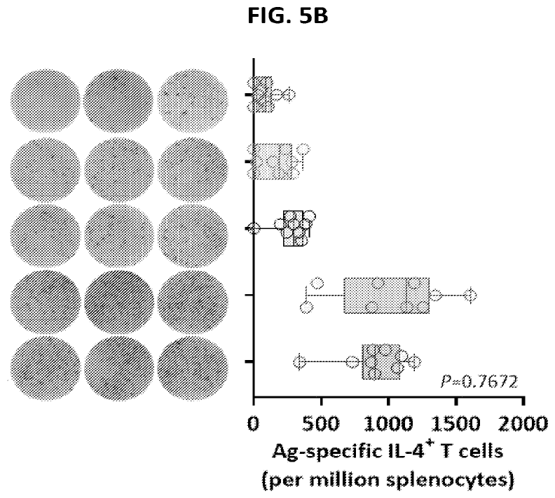

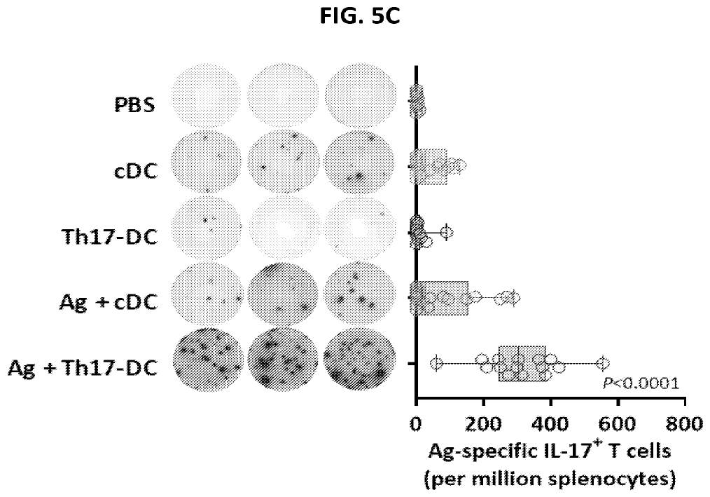

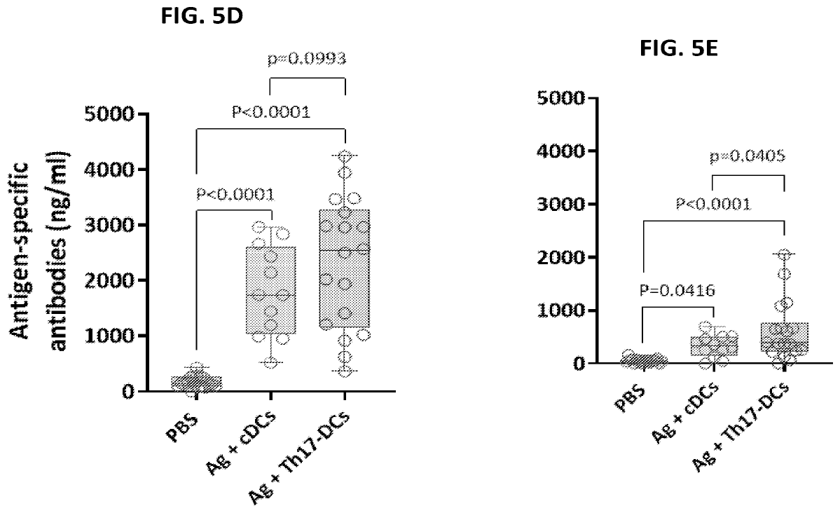

IL-15 costimulation and p38-MAPK blockade specifically empowers DC vaccines to generate IL-17⁺ T cells and high affinity antibodies in vivo, with little impact on generation of IFN-γ⁺ or IL-4⁺ T cells: To determine the ability of Th17 DCs to generate Th17 in vivo, DCs were prepared and either pulsed with tumor antigen or left unpulsed. ID8-SP17 tumor-bearing mice (minimal tumor load at one week post tumor challenge) were vaccinated and IFN-γ⁺, IL-4⁺ and IL-17⁺ T cells were measured two weeks following four weekly immunizations. Both antigen-pulsed conventional DCs (cDCs) and Th17 DCs led to marked and statistically significant increases in the numbers of antigen-specific IFN-γ⁺ and IL-4⁺ T cells, which were not evident when antigen was omitted (FIGS. 5A and 5B). Only antigen-pulsed Th17 DCs reliably led to the generation of tumor antigen-specific IL-17⁺ T cell immunity (FIG. 5C). Levels of vaccine-induced tumor-specific antibodies were strongly induced in the serum but the levels were not different between cDCs and Th17 DC vaccines (FIG. 5D). However, assay of antibodies under strong dissociation conditions (i.e., 6M urea) revealed that the Th17 DC vaccines tended to induce a higher avidity antibody repertoire (FIG. 5E). Overall, these results suggested that IL-15 co-stimulation and p38 MAPK blockade leads to highly selective modifications of DCs that specifically result in the activation of antigen-specific Th17 T cells and increased antibody avidity, with little impact on Th1 and Th2 T cell frequencies.

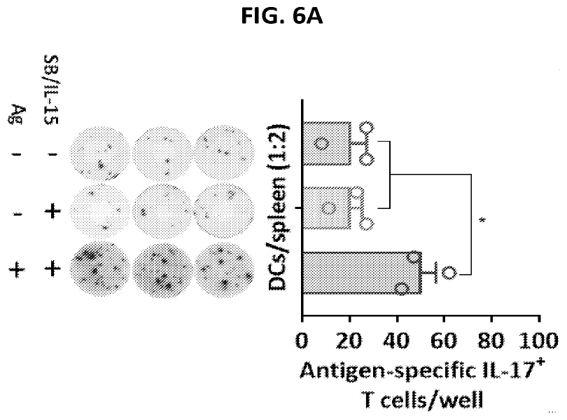

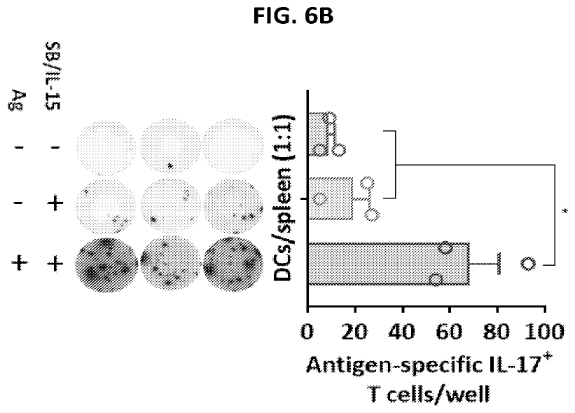

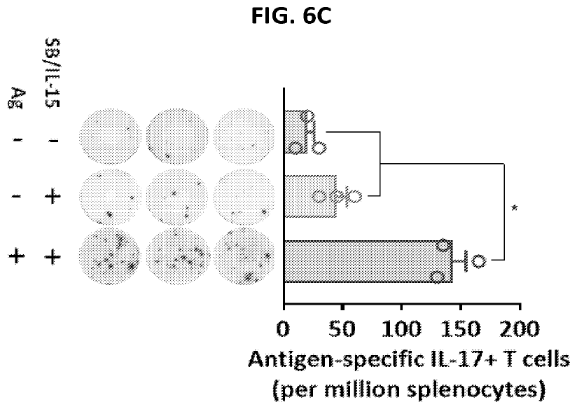

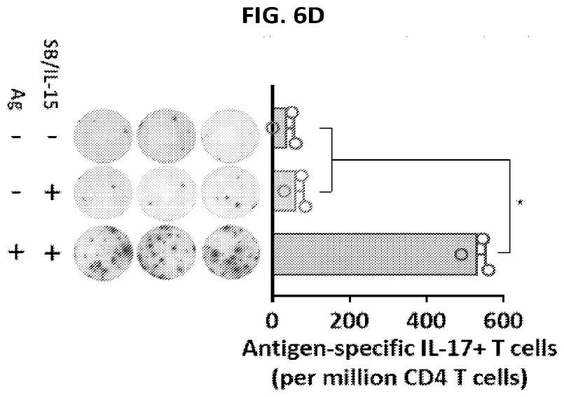

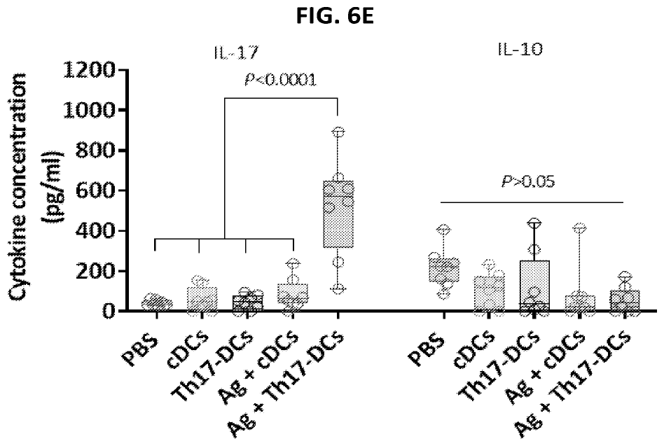

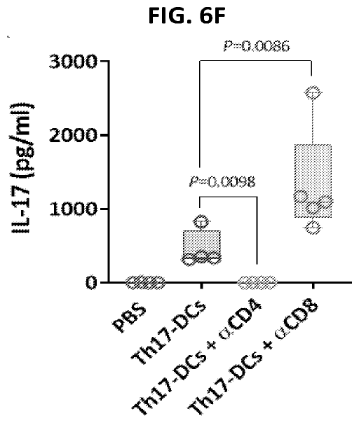

IL-15 costimulation and p38-MAPK blockade specifically empowers DC vaccines to rapidly generate antigen-specific Th17 T cells in vitro and in vivo: To determine if Th17-inducing DCs directly induce IL-17⁺ T cells, in vitro experiments were done using Th17 DCs to stimulate splenocytes derived from naïve non-tumor-bearing mice. As shown in FIGS. 6A and 6B, Th17 DCs were able to rapidly generate antigen-specific IL-17⁺ T cells within 72 hours from freshly prepared splenocytes. To ascertain that the antigen-specific IL-17⁺ T cells generated in vivo were CD4 T cells, splenocytes were harvested from immunized mice and CD4 T cells were magnetically purified followed by re-stimulation with antigen ex vivo. As demonstrated in FIGS. 6C and 6D, CD4 T cells fractionated directly from splenocytes of immunized mice were highly enriched in antigen-specific Th17 T cells. Further analysis showed that vaccination specifically led to high levels of circulating IL-17 but not IL-10 in tumor-bearing animals following vaccination (FIG. 6E) demonstrating that the vaccination likely did not result in Treg17 or rTh17 cells as reported elsewhere in small intestine and other tissues (Esplugues et al., Nature 475:514-518, 2011). In addition, as further demonstration that CD4 T cells were the source of IL-17 in vivo, mice were immunized as in FIG. 6E with antigen-pulsed Th17 DCs with or without CD4 or CD8 T cell depletion. As shown in FIG. 6F, depletion of CD4 T cells but not CD8 T cells eliminated IL-17 release. It also was noted that, unexpectedly, depletion of CD8 T cells resulted in statistically elevated levels of IL-17, suggesting that CD8 T cells can negatively modulate Th17 T cell induction activity in vivo. These results demonstrated that IL-15 costimulation and p38 blockade in DCs resulted in rapid priming and expansion of CD4⁺ Th17 T cells and not CD8⁺ Tc17 T cells that have been reported elsewhere (Hamada et al., J Immunol 182:3469-3481, 2009).

Th17 DC-induced T cell immunity elicits a unique myeloid cytokine signature: To determine if Th17 DC induced T cell immunity was associated with a unique cytokine/chemokine profile, proteomic analysis (TABLE 1) was performed on media derived from co-cultures of tumor-antigen-loaded cDCs incubated with purified CD4 T cells from mice immunized four times with either PBS, cDCs, or Th17 DCs. As shown in FIG. 7, Th17 DC vaccine induced CD4 T cells capable of stimulating elevated levels, compared to T cells from cDC vaccination, of various myeloid cell modulating cytokines/chemokines, including CCL6, CX3CL1, CXCL16, GM-CSF, ICAM-1, IL-1a, LIX, Osteoprotegerin and RBP-4 (adjusted P<0.05). Thus, vaccination with Th17 DC vaccines led to a distinct cytokine profile following interaction of vaccine-elicited T cells with antigen-presenting cells.

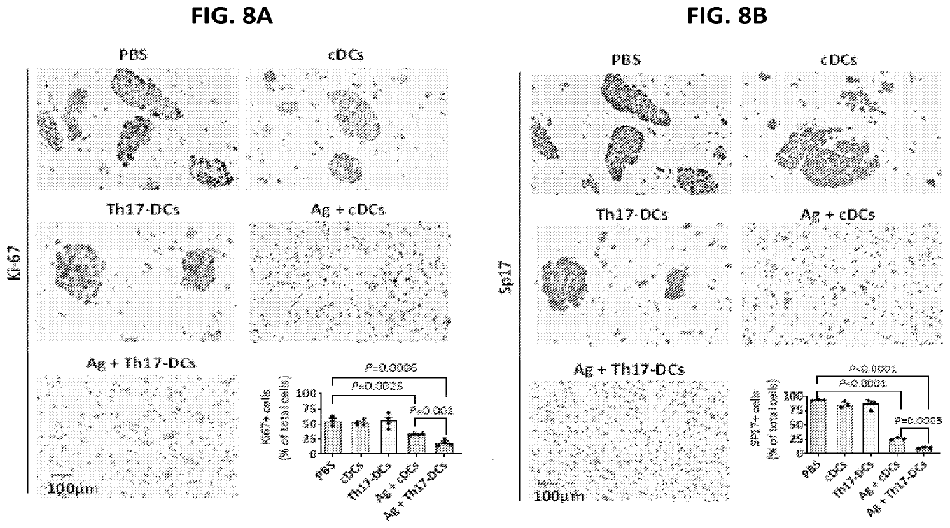

Th17-inducing vaccines eliminate shedding into the peritoneal cavity: Ovarian cancer is unique among other cancers in that metastasis is mediated through exfoliation and accumulation of tumor cells that can subsequently seed the omentum, the peritoneum, and abdominal organs (Lengyel, Am J Pathol 177:1053-1064, 2010). To determine if Th17 DC vaccines can impact accumulation of tumor cells, peritoneal tumor burden was analyzed with Ki-67 and Sp17 IHC staining of peritoneal cavity-derived exfoliated cells following tumor challenge. FIGS. 8A and 8B show representative images and quantitative analyses of Ki-67 and Sp17 tumor marker staining (inset graphs) in peritoneal cavity cells. A significant observation was the suppression of formation of large tumor cell clusters in mice immunized with antigen-pulsed vaccine. While the clusters were abundant in control animals, they were not usually observed in samples from vaccinated mice. The numbers of positive Ki-67 or Sp17 staining cells (both in clusters and in single cells) were sharply decreased in animals immunized with either antigen-pulsed cDCs or antigen-pulsed Th17 DCs. Th17 DCs appeared superior to cDC vaccines using both assessments. Decreases were entirely dependent on the presence of tumor antigen (Ag) in the vaccine, and were not evident with unpulsed cDCs or Th17 DCs. Thus, Th17 DC vaccines can reduce ovarian cancer-associated carcinomatosis.

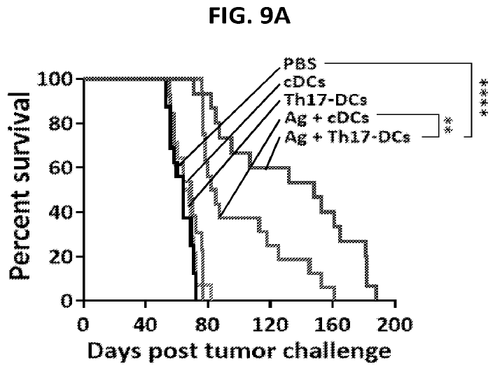

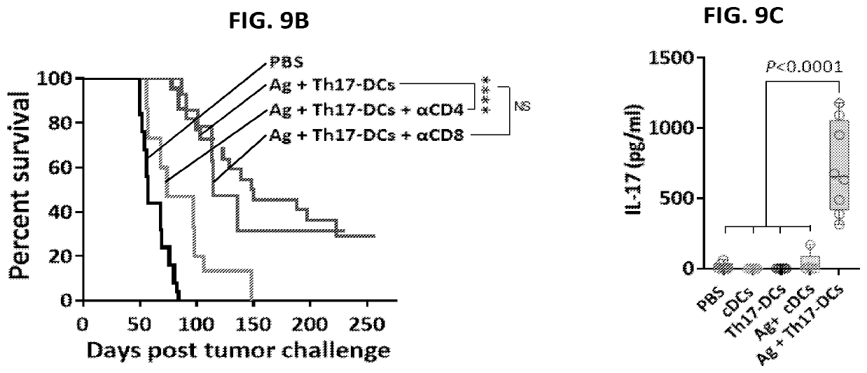

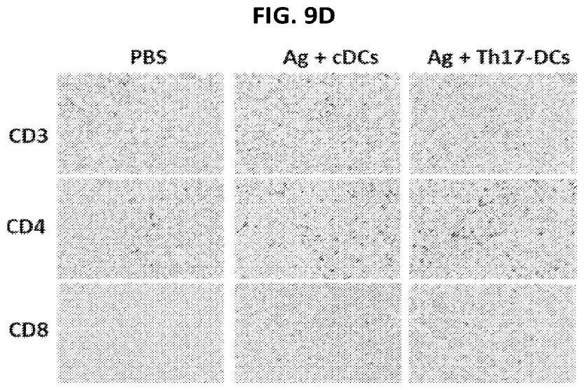

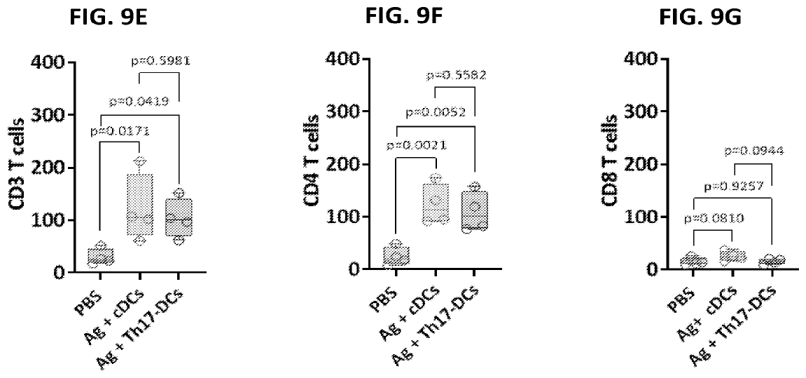

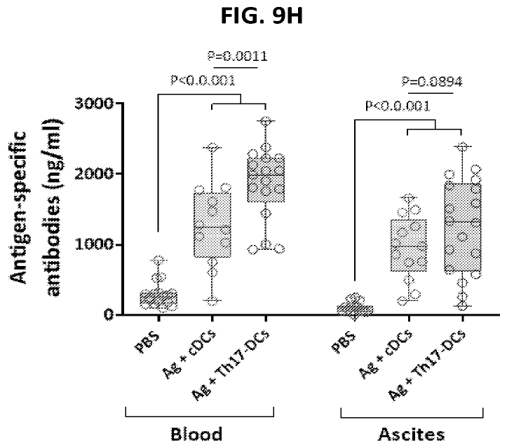

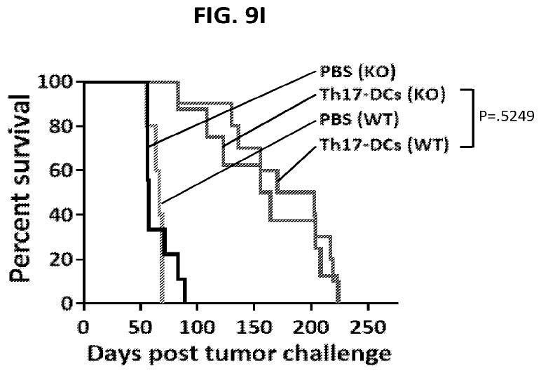

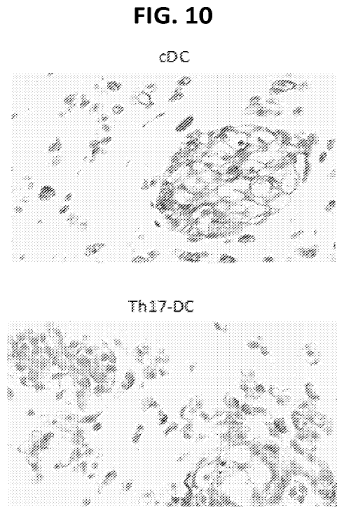

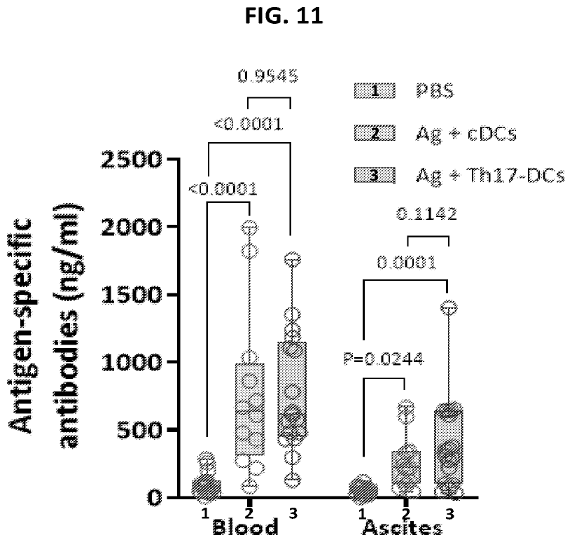

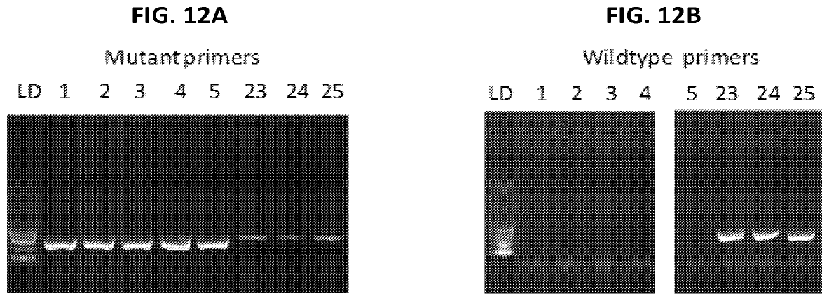

Th17 DC vaccination induces infiltration of T cells into solid tumor tissue and extends the lifespan of mice bearing ovarian cancer: To assess the impact of Th17 vaccines on survival, tumor-bearing mice were immunized as and followed until moribund. Tumor-bearing mice in the PBS-, unpulsed cDC-, and unpulsed Th17 DC-vaccinated groups succumbed to disease from days 60-80. However, mice immunized with either antigen-pulsed cDCs or antigen-pulsed Th17 DCs displayed prolonged survival (p<0.0001) (FIG. 9A). Antigen-pulsed Th17 DCs were superior to antigen-pulsed cDC vaccines, with a median survival of 148 days as compared to 84 days (p<0.01). The survival advantage imparted by the Th17 DC vaccine was reversed with depletion of CD4 T cells but not CD8 T cells (FIG. 9B). Ascites fluid derived from moribund animals showed accumulation of IL-17, suggesting that outgrowth was due to evasion strategies and not loss of Th17 immunity (FIG. 9C). Furthermore, tumor tissue in the peritoneal cavity at the times when Th17 DC vaccinated mice were moribund revealed high-level infiltration of CD4 T cells but relatively sparse CD8 T cell infiltration with either cDCs or Th17 DCs (FIGS. 9D-9G). Tumors also were observed to express MHC class II (FIG. 10). Although levels of antigen-specific antibodies were higher in blood upon death in Th17 DC vaccinated mice as compared to mice vaccinated with cDCs, similar levels were observed in the ascites (FIG. 9H). The increased levels of antibody in the blood were likely due to the differential collection times. By the time animals were moribund, the avidities of the antibody repertoire were similar amongst animals treated with cDCs and Th17 DCs (FIG. 11). To determine if IL-17A cytokine was required for the Th17 DC vaccine efficacy, survival was examined in tumor-bearing Il17a^{cre} mice, which have a Cre-Recombinase knocked in at the endogenous Il17a gene, abolishing IL-17a expression (FIGS. 12A and 12B). As shown in FIG. 9I, elimination of IL-17A had no impact on vaccine efficacy, suggesting that other cytokines released by Th17 are more important. Thus, Th17 DC vaccination induces durable tumor-infiltrating immunity and improves survival to an extent greater than that of the cDC vaccine, in a CD4 T cell-dependent and IL-17-independent manner. Ultimately, however, the mice succumb due to development of a yet unknown immune escape mechanism.

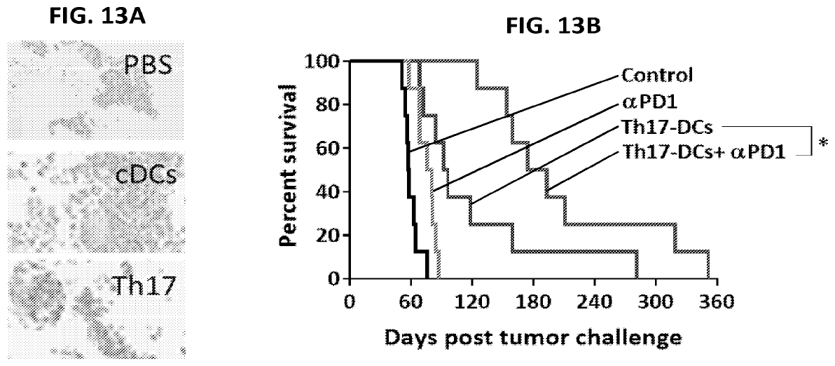

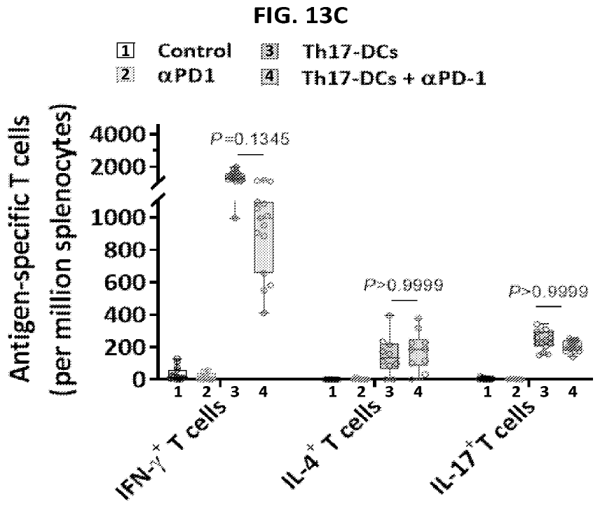

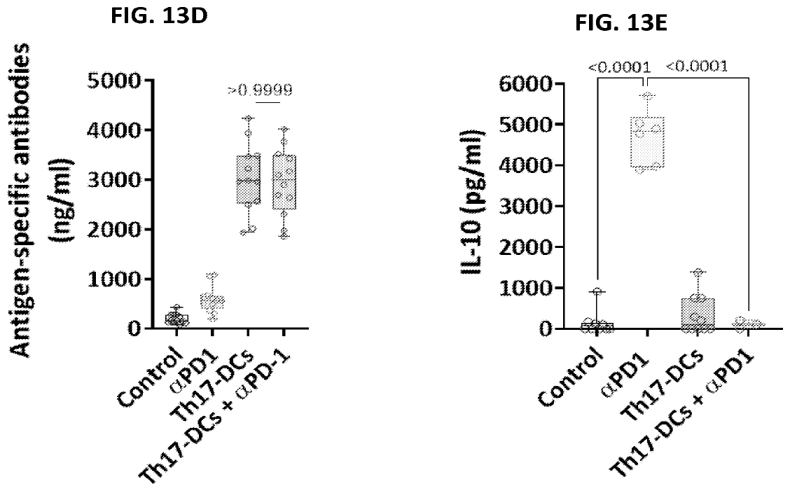

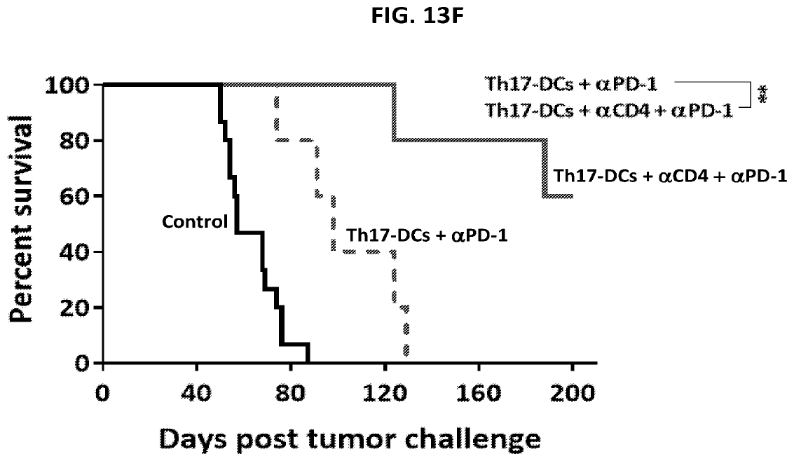

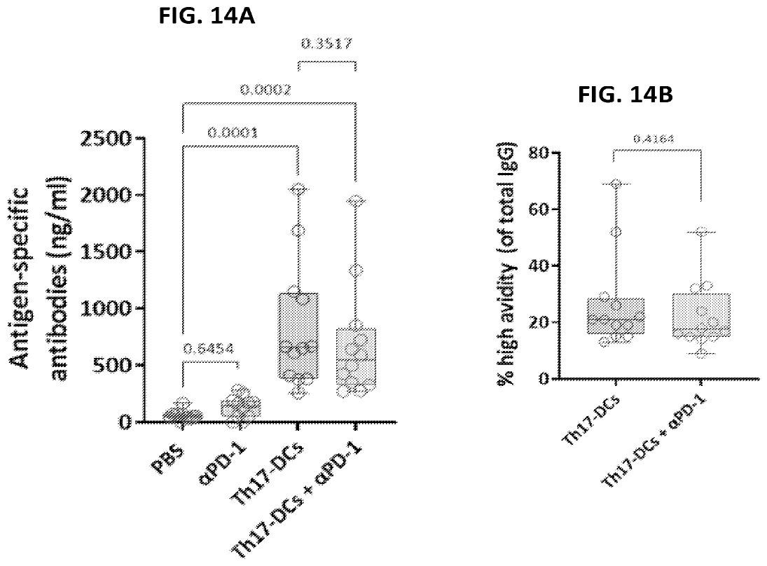

Th17 DC vaccination synergizes with immune checkpoint blockade in a CD4 T cell dependent manner: While Th17 vaccination can significantly improve survival in tumor-bearing mice, the mice (with rare exceptions) generally succumbed to disease. Studies described elsewhere suggested that the PD-1/PD-L1 axis is a potentially important immune regulatory pathway in ovarian cancer despite the observations that specifically targeting the pathway with immune checkpoint blockade molecules in the clinic had limited results (Hamanishi et al., J Clin Oncol 33:4015-4022, 2015; Matulonis et al., Ann Oncol 30:1080-1087, 2019; Krempski et al., J Immunol 186:6905-6913, 2011; Lamichhane et al., supra, and Wang, J Ovarian Res 12:37, 2019). To investigate whether the axis regulated the activity of the vaccination or vice versa, periodic anti-PD-1 injections were included during the vaccination in tumor-bearing mice. ID8-SP17 cells expressed PD-L1 in vivo, in both control animals and in animals treated with cDC and Th17 DC vaccines (FIG. 13A). FIG. 13B shows that PD-1 therapy alone had little activity, with only a marginal increase in survival relative to no treatment, which was consistent with our observations described elsewhere (Krempski et al., supra, Lamichhane et al., supra). Although statistically significant (p=0.003), survival was incrementally improved by only ~20 days (78 days in the PD-1 group vs. 58 days in the PBS group). In contrast, the addition of anti-PD-1 nearly doubled the median survival as compared to Th17 DC vaccination alone (p=0.02). One possible explanation for the augmented survival observed by the addition of anti-PD-1 antibody treatment is that it increased the number of antigen-specific effectors induced by vaccine. ELISpot analysis, however, revealed that the T cell immune responses were similar or perhaps slightly reduced (e.g., Th1) by anti-PD-1 treatment (FIG. 13C). Similarly, inclusion of anti-PD-1 did not alter levels or avidity of Th17 DC induced antibodies (FIGS. 13D, 14A, and 14B).

In studies described elsewhere (Lamichhane et al., supra), it was observed that IL-10 production was strongly induced within the ovarian cancer microenvironment upon administration of single agent anti-PD-1 that sustains local immune suppression of T cells responses (Lamichhane et al., supra). In that study, blockade of IL-10 with anti-IL-10 and anti-IL-10 receptor antibodies along with PD-1 led to improved survival, demonstrating that IL-10 release is a major mediator of adaptive resistance following anti-PD-1 treatment. Studies were conducted to determine whether IL-10 was present in the tumor microenvironment (ascites) when animals were moribund revealed that IL-10 was highly elevated in the ascites in mice that received only anti-PD-1 antibody but not when the vaccine was given in combination (FIG. 13E). Thus, Th17 vaccination overcame IL-10-mediated resistance to anti-PD-1 therapy in the peritoneal cavity.

To specifically determine the role of the CD4 T cell responses in the efficacy of Th17 DC vaccination, either alone or in combination with anti-PD-1, T cells were depleted with anti-CD4 monoclonal antibodies during vaccination and immune checkpoint blockade therapy. Depletion was maintained with weekly booster injections of anti-CD4 antibody. As shown in FIG. 13F, the inclusion of anti-CD4 significantly, albeit not completely, reversed protection afforded by combination Th17 DC vaccination and anti-PD-1, demonstrating a clear role of CD4 T cells in the efficacy of the treatment.

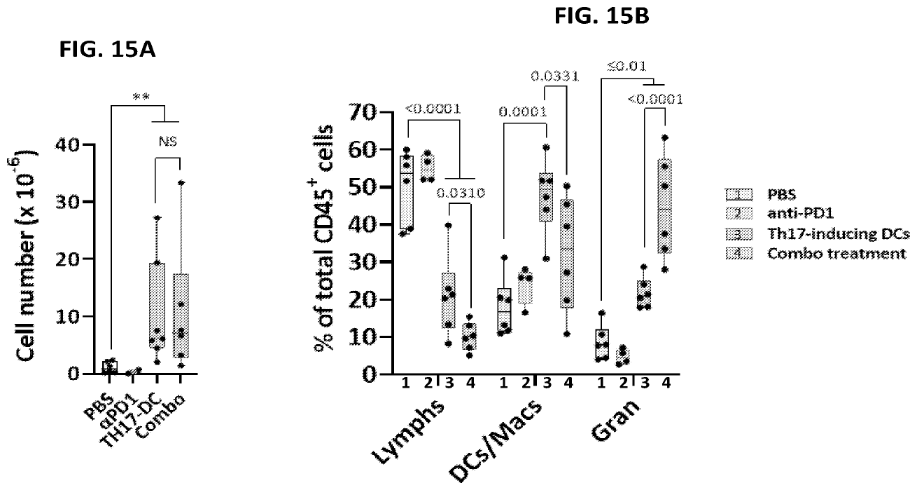

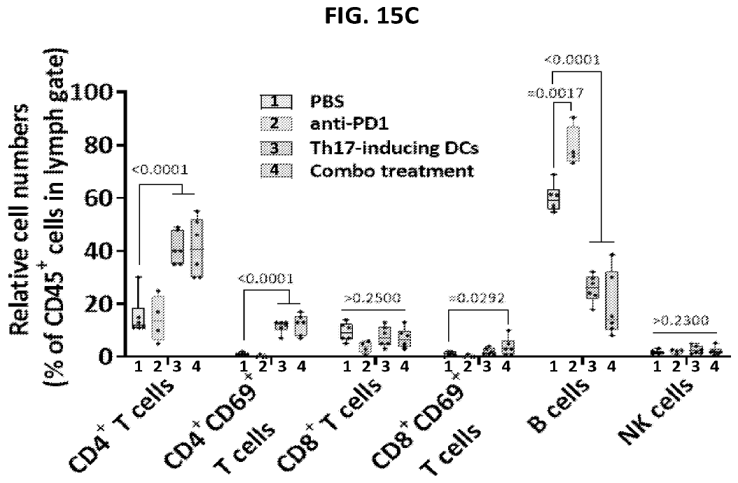

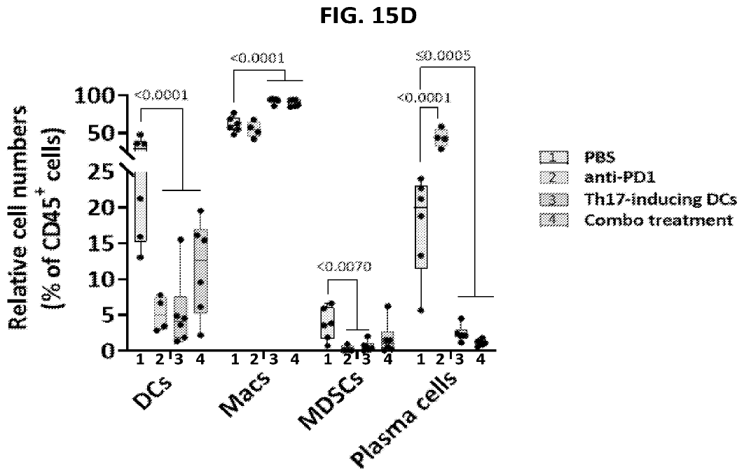

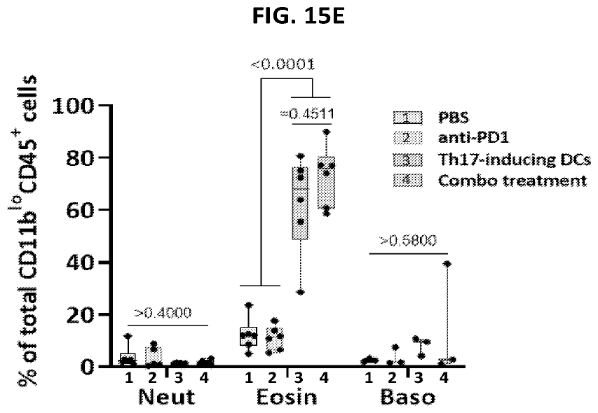

Th17 DC vaccination alone or in combination results in CD4 T cell driven macrophage and eosinophil infiltration into the peritoneal cavity of tumor-bearing mice: Understanding the tumor immune microenvironment during treatment can be useful in several ways, including identifying mechanisms of action and biomarkers. Since increased immunity was not observed due to inclusion of anti-PD-1, the peritoneal cavity tumor immune microenvironment of treated and untreated mice was examined in further detail. Treatment of tumor-bearing mice with monotherapy or combination therapy led to measurable shifts in cell number and immune cell infiltration into the peritoneal cavity at day 42 following 4 vaccinations, as shown in FIGS. 15A-15E. Vaccine was required to induce infiltration into the peritoneal cavity, and anti-PD-1 had little effect on the number of immune cells harvested from the cavity (FIG. 15A). In both control (PBS) and anti-PD-1-treated mice, lymphocytes were the dominant immune effector population (~50-60%), followed by monocytes (DCs and macrophages, ~20%) and a small granulocyte fraction (~5-10%) (FIG. 15B). Immunization with Th17 DC vaccine markedly shifted the distribution, reducing the proportion of lymphocytes (~20%) and increasing the proportions of monocytes and granulocytes to ~50% and 20%, respectively. The inclusion of anti-PD-1 with vaccine further reduced the lymphocyte fraction to ~10% and markedly increased the granulocytes to ~40-45%.

Finer analysis of the lymphocyte fraction showed that the native lymphocyte response (in control mice) was largely constituted by B cell infiltration (~60%), whereas CD4 (15-20%) and CD8 (~10%) T cells constituted a small fraction. Treatment of mice with single agent anti-PD-1 slightly increased the fraction of B cells, with minimal effect on the proportion of T cells (either total or activated) (FIG. 15C). Treatment of mice with Th17 DC vaccine significantly increased the proportion of total and activated CD4 T cells and reduced the proportion of B cells. It was notable that that the inclusion of anti-PD-1 along with vaccine did not alter this distribution. Proportions of CD8 T and NK cells, which constituted a very minor fraction of the lymphocytes, were also not impacted with the vaccine or anti-PD-1, although there was a low-level increase in the number of activated CD8 T cells as assessed by CD69 expression. Thus, the major impact of vaccination overall, with or with anti-PD-1 therapy, was to shift lymphocyte immunity in favor of a CD4 T cell response and reduced B cell infiltration.

Given the observation that vaccine significantly induced shifts in the myeloid cell compartment, additional analysis was conducted to evaluate the monocyte gate by flow cytometry. In the absence of treatment, monocytes, which represented about 20% of the peritoneal infiltrate (FIG. 15B), were largely composed of DCs (~25-30%) and macrophages (~60%), with smaller components of MDSCs (~3-4%) and CD19″ putative plasma or activated B cells (~15%) (FIG. 15D). Treatment with anti-PD-1 alone reduced the proportion of DCs (~5%) and eliminated MDSCs (<1%) but led to a significant increase in the proportion of CD19⁺ plasma/activated B cells (from ~15% to 45%) while the proportion of macrophages remained similar. Th17 DC vaccinations alone or combined with anti-PD-1 significantly reduced the proportion of DCs (~5%), MDSCs (<1%) and CD19+ plasma cells (~2-3%), whereas the proportion of macrophages significantly increased to ~90-100% of the total monocytes. Thus, treatment with anti-PD-1 favored expansion of B cells while Th17 vaccination favored expansion of macrophages. With the combination approach, the effects of vaccine appear to prevail. While granulocytes were a minor fraction in control and anti-PD-1 monotherapy arms (<10%), vaccination alone or without anti-PD-1 resulted in a significant upregulation, particularly in the combination treatment group where granulocytes represented ~45% of the total immune cell infiltrate (FIG. 15B). Subsequent analysis of the granulocyte population revealed that the dominant cell type was eosinophils identified at Siglec-F+, CD193+ granulocyte, whereas Ly6G+ neutrophils and FceR1a+, CD200R3+ basophils were minor components.

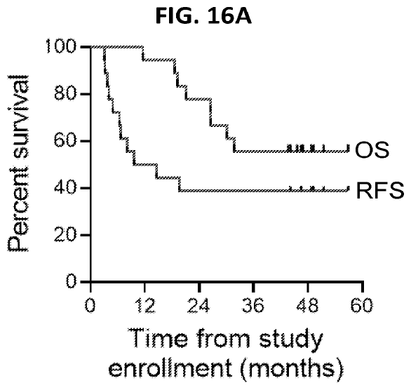

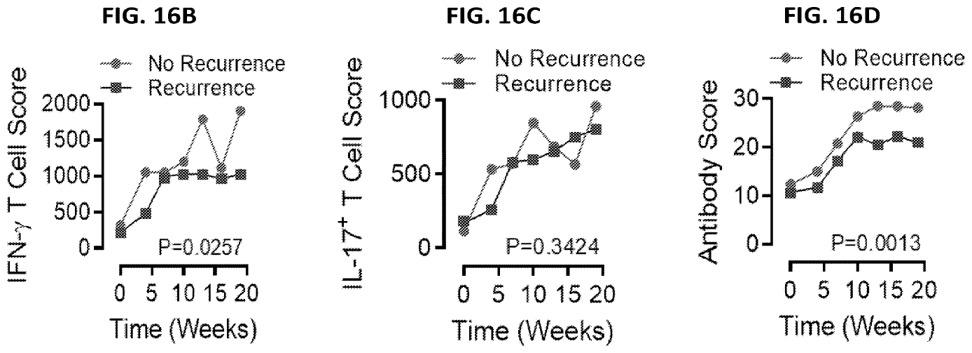

FRα-specific Th17 T cell responses can be safely generated in OC patients in the setting of minimal residual disease following conventional debulking surgery and adjuvant chemotherapy: A phase I trial was carried out to evaluate the safety and immunogenicity of Th17 DC vaccination in stage IIIC/IV OC patients in first remission after standard of care therapy. In contrast to the tumor lysates that were used in the murine studies, the clinical trial targeted folate receptor alpha (FRα), which is overexpressed 80- to 90-fold in more than 90 percent of OC (Li et al., J Nucl Med. 37 (4): 665-672, 1996). cGMP-grade autologous DCs were generated from peripheral monocytes with p38 MAPK inhibitor and IL-15, and were pulsed with five degenerate subdominant MHC class II epitopes from FRα (Kalli et al., Clin Cancer Res. 24 (13): 3014-3025, 2018). Five immunizations and up to 7 boosters were given intradermally. 19 patients were enrolled over the 16 months the trial was open. No grade ≥3 adverse events were seen. RFS and OS are shown in FIG. 16A. At the time of data cut-off, 38.9% of the patients remained alive and free from recurrence, and all patients who did not recur during the vaccine maintenance period remain recurrence free at present (median follow-up: 49.2 months). RFS compared much favorably to that observed in other recent trials including EORTC 55971 and CHORUS (~10% progression-free survival at 36 months following randomization) and the GOG-0218 bevacizumab phase III clinical trial (≤15% progression-free survival at 36 months following randomization) (Vergote et al., Lancet Oncol. 19 (12): 1680-1771, 2018; Burger et al., N Engl J Med. 365 (26): 2473-2483, 2011; and Perren et al., N Engl J Med. 365 (26): 2484-2496, 2011). There was no correlation between survival and MRD or BRCA1/2 mutation status.

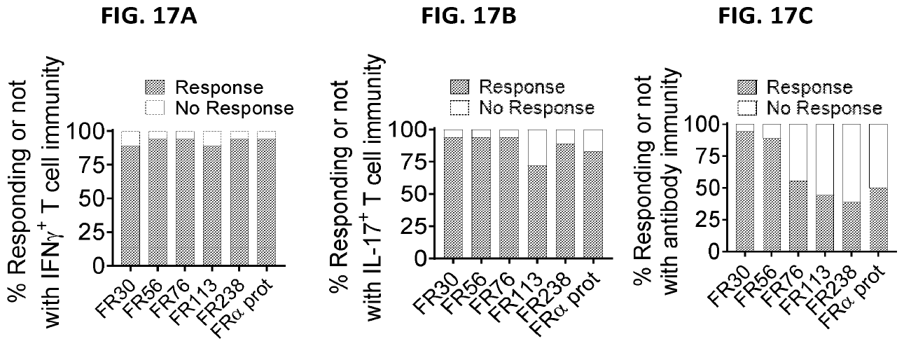

Vaccination triggered Th17, Th1, and antibody immunity to the constituent FRα epitopes, as well as to the whole FRα protein over the 19-week vaccination period; immunity was generally higher in patients who did not recur (FIGS. 16B-16D). Comparisons of pre- and post-vaccine T cell frequencies showed that increased frequencies of Th17 responses to FRα were elevated in 78% of patients (14/18), with responses to all individual epitopes seen in 72-94% of patients. Similarly, results were observed for Th1 cells specific for each FRα peptide and for the FRα protein, with 89% of patients (16/18) exhibiting Th1 cell responses to FRα. Th1 response rates to the five FRα peptides ranged between 89-100% (FIG. 17A-17C). Most patients demonstrated elevated antibodies to epitopes at one or more time points during immunization, and antibody responses targeting native FRα protein were observed in 50% of patients, with antibody responses to each of the vaccine peptides seen in 39-94% of patients. Levels of FRα-specific T cells and antibodies remained elevated relative to baseline throughout the period of study participation (weeks 27-104). This suggested that immunologic memory is induced.

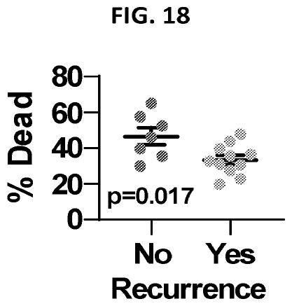

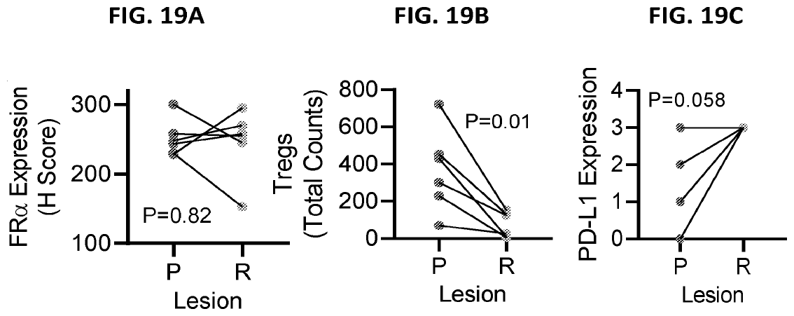

In addition to higher levels of T cell and antibody immunity in patients who did not relapse, patients without recurrence demonstrated higher levels of circulating cytotoxic IgG antibodies to native FRα protein relative to those that recurred (FIG. 18). Other pre-treatment biomarkers were compared among patients that recurred and those that were protected, revealing no significant differences in circulating Tregs, tumor-infiltrating Tregs, tumor-infiltrating CD8 T cells, tumor PD-L1 expression, and tumor FRα expression. Patients who did relapse did not demonstrate antigen-loss but did show some evidence that immunization impacted the tumor microenvironment by reducing the levels of Tregs and elevating the levels of PD-L1 expression by the tumor (FIGS. 19A-19C). These studies indicated that Th17-inducing DC vaccination is safe, induces antigen-specific immunity in most individuals, and associates with prolonged RFS in some patients.

In summary, the human data and the data from murine modeling suggested that (1) Th17 T cells coordinate otherwise ineffective Th1 and Th2 immunity and eliminate tumor independent of CD8 T cells and MHC class I; (2) Th17 T cells release myeloid mediators (MCP5 and LIF) that recruit macrophages and eosinophils to directly kill antibody-coated tumor cells; (3) Th17 T cells release CST3, which elevates levels of IFN-γ, sensitizing macrophages and eosinophils; (4) Th17 T cells release RBP4, which prevents M2 (immunosuppressive) skewing of macrophages; (5) Th17 DC vaccine leads to restructuring of the tumor immune microenvironment; and (6) Th17 DC vaccine prevents anti-PD-1 mediated adaptive resistance and sensitizes OC to ICB therapy. Given the results described herein, the combination of Th17 DC vaccine and ICB may be an effective therapeutic strategy to extend the life of women with recurrent ovarian cancer.

### Example 2—Th17 T Cell-Inducing Vaccines Subvert Resistance of Breast Cancer to Immune Checkpoint Blockade

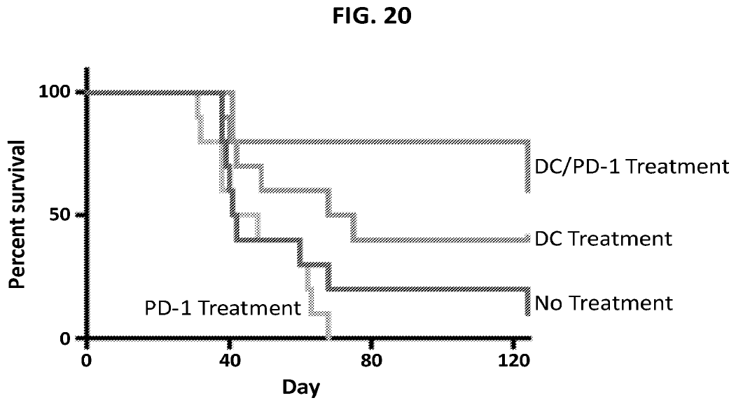

In further studies, mice with triple negative breast cancer (TNBC) were treated with an Sp17 antigen-loaded Th17-DC vaccine, anti-PD-1, or a combination of the antigen-loaded Th17-DC vaccine plus the anti-PD-1. SP17 is a TNBC tumor antigen. The mouse Sp17 antigen was formulated as a series of 32 amino acid peptides, overlapping by 10 amino acids and covering residues 1-142 of the Sp17 sequence (the full length sequence is 151 amino acids in length). Th17-DC were loaded with individual peptides at 50 μg/mL on day 5 (when maturation cytokines also were added), and were harvested and pooled on day 7. Mice were engrafted with 1×10⁵ E0771 TNBC subcutaneously in or around the mammary fat pad. Groups of mice were then treated with the Th17 DC vaccine (n=10), anti-PD-1 (n=10), the Th17 DC vaccine and anti-PD-1 (n=8), or nothing (n=10). Th17-DC vaccination (1×10⁶ cells administered intraperitoneally) was given every 7 days starting on day 7 after tumor engraftment for a total of 4 vaccinations, and anti-PD-1 (Bio X Cell) was administered intraperitoneally at a dose of 200 μg in 0.1 mL PBS, 3 and 5 days after each Th17-DC vaccination. Mice were sacrificed when their tumors reached >1 cm diameter. As shown in the Kaplan-Meier plot presented in FIG. 20, the mice treated with the combination therapy demonstrated significantly prolonged survival compared to the control (no treatment) group (p=0.0112, Mantel-Cox) and compared to the group treated with only anti-PD-1 (p=0.0005).

TABLE 1
Cytokines examined by dot blot analysis

| | | |
|---|---|---|
| Adiponectin/Acrp30 | DPPIV/CD26 | IL27p28 |
| Amphiregulin | EGF | IL-28A/B |
| Angiopoietin-1 | Endoglin/CD105 | IL-33 |
| Angiopoietin-2 | Endostatin | LDL R |
| Angiopoietin-like 3 | Fetuin A/AHSG | Leptin |
| BAFF/BLyS/TNFSF13B | FGF acidic | LIF |
| C1q R1/CD93 | FGF-21 | Lipocalin-2/NGAL |
| CCL2/JE/MCP-1 | Flt-3 Ligand | LIX |
| CCL3/CCL4/MIP-1α/β | Gas 6 | M-CSF |
| CCL5/RANTES | G-CSF | MMP-2 |
| CCL6/C10 | GDF-15 | MMP-3 |
| CCL11/Eotaxin | GM-CSF | MMP-9 |
| CCL12/MCP-5 | HGF | Myeloperoxidase |
| CCL17/TARC | ICAM-1/CD54 | Osteopontin (OPN) |
| CCL19/MIP-3β | IFN-γ | Osteoprotegerin/TNFRSF11B |
| CCL20/MIP-3α | IGFBP-1 | PD-ECGF/Thymidine phosphorylase |
| CCL21/6Ckine | IGFBP-2 | PDGF-BB |
| CCL22/MDC | IGFBP-3 | Pentraxin 2/SAP |
| CD14 | IGFBP-5 | Pentraxin 3/TSG-14 |
| CD40/TNFRSF5 | IGFBP-6 | Periostin/OSF-2 |
| CD160 | IL-1α/IL-1F1 | Pref-1/DLK-1/FA1 |
| Chemerin | IL-1β/IL-1F2 | Proliferin |
| Chitinase 3-like 1 | IL-1ra/IL-1F3 | Proprotein Convertase 9/PCSK9 |
| Coagulation Factor III/Tissue Factor | IL-2 | RAGE |
| Complement Component C5/C5a | IL-3 | RBP4 |
| Complement Factor D | IL-4 | Reg3G |
| C-Reactive Protein/CRP | IL-5 | Resistin |
| CX3CL1/Fractalkine | IL-6 | E-Selectin/CD62E |
| CXCL1/KC | IL-7 | P-Selectin/CD62P |
| CXCL2/MIP-2 | IL-10 | Serpin E1/PAI-1 |
| CXCL9/MIG | IL-11 | Serpin F1/PEDF |
| CXCL10/IP-10 | IL-12p40 | Thrombopoietin |
| CXCL11/I-TAC | IL-13 | TIM-1/KIM-1/HAVCR |
| CXCL13/BLC/BCA-1 | IL-15 | TNF-α |
| CXCL16 | IL-17a | VCAM-1/CD106 |
| Cystatin C | IL-22 | VEGF |
| DKK-1 | IL-23 | WISP-1/CCN4 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal having cancer, wherein said method comprises:
(a) administering, to said mammal, an effective amount of a vaccine composition capable of activating T cells within said mammal to form activated IL-17-secreting T cells that can recognize one or more antigens expressed by said cancer, and
(b) administering, to said mammal, an effective amount of an immune checkpoint inhibitor (ICI).

2. The method of claim 1, wherein said mammal is a human.

3. The method of claim 1, wherein said cancer is ovarian cancer, melanoma, chronic lymphocytic leukemia, gastric cancer, cervical cancer, colorectal cancer, breast cancer, or lung cancer.

4. The method of claim 1, wherein said vaccine composition comprises dendritic cells (DCs) presenting one or more folate receptor alpha (FRα) antigens.

5. The method of claim 4, wherein said one or more FRα antigens comprise one or more peptides having the amino acid sequence set forth in SEQ ID NO:3, SEQ ID NO:4, SEQ ID NO:5, SEQ ID NO:7, or SEQ ID NO: 14.

6. The method of claim 1, wherein said method comprises administering a dose of about 5×10⁶ to about 50×10⁶ of said DCs composition to said mammal.

7. The method of claim 1, wherein said method comprises administering said vaccine composition to said mammal two or more times over a time period of about 2 weeks to about 16 weeks.

8. The method of claim 1, wherein said ICI is selected from the group consisting of pembrolizumab, atezolizumab, nivolumab, durvalumab, avelumab, tremelimumab, ipilimumab, and cemiplimab.

9. The method of claim 1, wherein said method comprises administering a dose of about 50 mg to about 1500 mg of said ICI to said mammal.

10. The method of claim 1, wherein said method comprises administering said ICI to said mammal two or more times over a time period of about 2 weeks to about 16 weeks.

11. The method of claim 1, wherein said method further comprises monitoring said mammal after said administering steps (a) and (b) to determine an effect of said vaccine composition and said ICI on said mammal.

12. The method of claim 11, wherein said monitoring comprises using computed tomography, positron emission tomography/computed tomography, bone scan, or magnetic resonance imaging.

13. The method of claim 12, wherein said monitoring comprises measuring the level of one or more biomarkers in a tumor biopsy obtained from said mammal after said administering steps (a) and (b), wherein said one or more biomarkers are selected from the group consisting of CCL6, CX3CL1, CXCL16, GM-CSF, ICAM-1, IL-1a, LIX, osteoprotegerin, and RBP-4.
